(19) United States
(12) Patent Application Publication
(10) Pub. No.: US 2018/0065859 A1
(43) Pub. Date: Mar. 8, 2018

(54) SILICA NANOSTRUCTURES, LARGE-SCALE FABRICATION METHODS, AND APPLICATIONS THEREOF

(71) Applicant: The Regents of the University of California, Oakland, CA (US)

(72) Inventors: Andrew C. Kummel, San Diego, CA (US); Alexander Liberman, San Diego, CA (US); Robert Viveros, San Diego, CA (US); William C. Trogler, Del Mar, CA (US)

(73) Assignee: The Regents of the University of California, Oakland, CA (US)

(21) Appl. No.: 15/559,764

(22) PCT Filed: Mar. 21, 2016
(86) PCT No.: PCT/US16/23492

§ 371 Date: Sep. 19, 2017

Related U.S. Application Data
(60) Provisional application No. 62/135,653, filed on Mar. 19, 2015.

(51) Int. Cl. C01B 33/16; A61K 49/18; H01G 4/08; C01B 33/027; C01B 33/146

(52) U.S. Cl. CPC C01B 33/16; A61K 49/1824; B82Y 40/00; C01B 33/027; C01B 33/146; H01G 4/08

(57) ABSTRACT

Methods, systems, and devices are disclosed for fabricating and utilizing nanoscale material structures such as nanoflakes and nanoshells.

### PRIORITY CLAIM AND RELATED APPLICATION

This application claims the benefits and priority of U.S. Provisional Application No. 62/135,653 entitled “SILICA NANOSTRUCTURES AND LARGE-SCALE FABRICATION METHODS” filed on Mar. 19, 2015, the entire disclosure of which is incorporated by reference as part of this application.

### STATEMENT REGARDING FEDERALLY SPONSORED RESEARCH OR DEVELOPMENT

This invention was made with government support under grants 1F31CA174276-01A1 and 1R33CA177449-01A1 awarded by the National Institutes of Health (NIH) along with grant 3R25CA153915 awarded by the NIH-Cross Training Translation Cancer Researchers in Nanotechnology (CRIN). The government has certain rights in the invention.

### TECHNICAL FIELD

This patent document relates to systems, devices, and processes to design, produce and utilize nanoscale materials.

### BACKGROUND

Nanotechnology provides techniques or processes for fabricating structures, devices, and systems with features at a molecular or atomic scale, e.g., structures in a range of one to hundreds of nanometers in some applications. For example, nano-scale devices can be configured to sizes within one hundred to ten thousand times smaller than human cells, e.g., similar in size to some large biological molecules (biomolecules) such as enzymes and receptors. Nano-sized materials used to create a nanostructure, nanodevice, or a nanosystem can exhibit various unique properties that are not present in the same materials scaled at larger dimensions and such unique properties can be exploited for a wide range of applications.

### SUMMARY

Disclosed are methods and materials pertaining to silica nanostructures and versatile, large-scale manufacture methods to fabricate silica nanostructures.

The subject matter described in this patent document can be implemented in specific ways that provide one or more of the following features. For example, the disclosed technology includes silica nanomaterials with engineered structures including nanoshells and nanoflakes. The disclosed methods include a variety of nanoscale fabrication techniques, including, for example, a technique for facile large-batch doping of sol-gel synthesized particles; a technique for scale-up synthesis of silica nanoshells based on stirring; a low-cost, aminated-silane-assisted template technique to synthesize silica nanoshells; and a scalable technique to synthesize tunable nanoflakes and nanoshells assembled from nanoflakes. The disclosed nanostructures are useful in a variety of applications, such as ultrathin silica nanoshells for ultrasound contrast agents, and silica nanoflakes as interlayer dielectric in graphene-insulator-graphene devices.

In some implementations, the disclosed the technology can be used to provide a method for large scale nanomaterial fabrication. This method includes synthesizing a silica nanoshell or nanoflake by doping sol-gel synthesized particles with either dopant sol gel precursors or nanoscales sols derived from them to produce silica nanostructures as described in this patent document; aminated-silane templating to produce silica nanoshells as described in this patent document; or assembling nanoflakes on templates in solution to form hollow nanoparticles, and isolation of individual nanoflakes as described in this patent document. In another implementation, the disclosed technology can be used to provide a method for large scale fabrication of doped nanoparticles that synthesizes nanoparticles using a sol-gel process; and then incubates the synthesized nanoparticles in a concentrated solution of the metal dopant at a plurality of temperatures with continuous mixing.

The disclosed technology can be used to provide a method of fabricating a hollow silica nanoshell to include mixing a particle template with a polyamine polymer and a silica precursor in a solution to coat the particle with a silica shell; adding one or more metal salts or metal oxides in the solution such that the metal is diffused into the silica shell of the particle; and calcinating the particle of form a hollow silica nanoshell. Various implementations are possible for the above method. For example, the polyamine polymer can be poly-L-lysine; the polyamine polymer can be poly-aminated silane N₁-(3-Trimethoxysilylpropyl) diethylenetriamine (DETA); the particle template can be a polystyrene template; the silica precursor is tetramethyl orthosilicate (TMOS); the silica precursor can include tetramethyl orthosilicate (TMOS) and one or more R-substituted trialkoxysilanes; the metal oxide can be iron ethoxide; the size of the hollow silica nanoshell can be between 500 nm and 2000 nm.

The disclosed technology can also be used to provide a method of fabricating a solid sol-gel nanoparticle to include mixing a silica precursor, in the absence of any template, in an ammonia solution containing ethanol to form a silica particle; adding one or more metal salts or metal oxides in the solution such that the metal is diffused into the silica particle; and calcinating the particle of form a solid sol-gel nanoparticle. Various implementations are possible for the above method. For example, the method can include adding a quenching agent when the silica particle grows to a desired size. For another example, the quenching agent can be a chemical capping agent.

In addition, the disclosed technology can be used to provide a method of fabricating doped nanoparticles synthesizing nanoparticles using a sol-gel process; and incubating the synthesized nanoparticles in a concentrated solution comprising one or more metal dopants at a plurality of temperatures with continuous mixing.

The above and other aspects, and associated implementations are described in greater detail in the description, the drawings and the claims.

### BRIEF DESCRIPTION OF THE DRAWINGS

FIG. 1 shows examples of molecular structures and naming scheme of R-group substituted trialkoxysilanes used in the modified sol-gel synthesis.

FIG. 2 shows examples of structures that are known to be successful templating agents. A) Poly-1-Lysine, MW˜150,000-300,000 Da; B) Polyethylenimine, MW˜20,000 Da; C) DETA, MW˜265 Da.

FIG. 3A shows an example of a process for performing Metal Doping of Hollow Sol-Gel Shells.

FIG. 3B shows an example of a process for performing Metal Doping of Solid Sol-Gel Particles.

FIG. 4 shoes examples of fabrication schemes of nanoflakes coating of semiconductor.

FIG. 5 shows examples of TEM images of all nanoshell formulations. All images were taken at 40,000× magnification and then 150,000× magnification for high resolution images of the shells. A-B) Control nanoshells. C-D) Nanoshells synthesized with C8. E-F) Nanoshells synthesized with C8-F. G-H) Nanoshells synthesized with phenyl. I-J) Nanoshells synthesized with phenyl-F. The nanoshells synthesized with C8 and phenyl had the thinnest walls.

FIG. 6 shows examples for showing theeffect of increasing TMCS. As the amount of 1% TMCS was increased going from 10 μL to 50 μL resulted in decreased pellet collection indicating that all of the reacted silane was staying in solution.

FIG. 7 shows two tests for the Tyndall effect of nanoflakes. A green laser was shown through (A) Pure Ethanol and (B) Ethanol containing nanoflakes (4-hour reaction time). The nanoflake containing solution exhibits a much stronger scattering of the laser beam, which results in a much brighter beam through the clear solution.

FIG. 8 shows examples of TEM images of nanoflakes. A) TEM of sol-gel reaction quenched at 2 hours. B) TEM of sol-gel reaction quenched at 4 hours.

FIG. 9 shows photographs of experiments under different mixing conditions of scaled up reactions. A) For 100-300×scale reactions, an oversized stir bar is used which generates increased shear mixing, a smaller sized round bottom flask forces the fluid to fold over its self-generating even more mixing. The mixing at 1200 RPM is sufficient to create a vortex effect and expose the center of the stir bar. B) For 500× or larger scale reactions a high shear mixer is used with a fixed impeller head. The impeller speed is typically between 3000-4000 rpm, which is sufficient to generate a vortex even with 500 ml of solution.

FIG. 10 shows examples of scale up images of 500 nm and 200 nm Nanoshells. A) 100× Scale Up of 500 nm Nanoshells synthesized with PEI templating. B) 300× Scale Up of 500 nm Nanoshells synthesized with PEI templating. C) 500× Scale up of 500 nm nanoshells synthesized with DETA templating.

FIG. 11 shows sample images of initial small scale synthesis of DETA templated particles. A) 30 μL of DETA added. B) 40 μL of DETA added. C) 80 μL of DETA added.

FIG. 12 shows sample images for incorporation of iron ethoxide and organically modified silanes into DETA templating synthesis of nanoshells. A) 80 μL of 0.2% DETA solution, trimethoxy(phenyl)silane: TMOS molar ratio is 55:45. Atomic % Fe by EDX =5.93 ±0.67%. B) 40 μL of 0.2% DETA solution, triethoxy(octyl)silane: TMOS molar ratio is 50:50. Atomic % Fe by EDX=5.62±0.29%.

FIG. 13 shows sample SEM imaging of gadolinium doped 2 micron silica shells. Samples A-C were treated at 50 C and samples D-F were treated at 75 C. A) Starting GdCl₃ solution concentration 5 mg/ml. B) Starting GdCl₃ solution concentration 10 mg/ml. C) Starting GdCl₃ solution concentration 20 mg/ml. D) Starting GdCl₃ solution concentration 5 mg/ml. E) Starting GdCl₃ solution concentration 10 mg/ml. F) Starting GdCl₃ solution concentration 20 mg/ml.

FIG. 14 shows sample SEM images of 500 nm particles incubated for 48 hours at 75 C in GdCl₃ solution. (A) 10 mg/ml, EDX analysis showed 13.36+/−5.84 Wt. % Gd (B) 20 mg/ml solution, EDX showed 18.26+/−1.60 Wt. % Gd.

FIG. 15 shows examples of iron doping of 500 nm particles for 72 hours at 75 C with iron (III) ethoxide/ethanol solution. A) 5 mg/ml, EDX was 14.54+/−1.58 Wt. % Fe B) 10 mg/ml, EDX was 35.44+/−3.11 Wt. % Fe C) 20 mg/ml, EDX was 43.46+/−3.87 Wt. % Fe.

FIG. 16 shows examples of iron doping of Stober synthesized 200 nm nanoparticles. Stage 1 synthesis was performed at room temperature before performing Stage 2 at 50° C. A) SEM of silica particles without dopant. B) The same particles after treatment in 10 mg/ml iron (III) ethoxide solution for 24 hours at 50° C. C) The silica particles without dopant on the left (white tube) and with iron doping on the right (brown tube).

FIG. 17 shows examples of Bismuth doping of 500 nm silica nanoshells. Particles were incubated for 48 hours at 75° C. with mixing at 1200 rpm then washed and calcined. A) Bismuth chloride concentration at 5 mg/ml B) Bismuth chloride concentration at 10 mg/ml C) Bismuth chloride concentration at 20 mg/ml.

FIG. 18 shows examples of manganese doping of 2 micron microshells. Particles were incubated for 48 hours at 50° C. with mixing at 1200 rpm then washed and calcined. A) Manganese chloride concentration at 5 mg/ml B) Manganese chloride concentration at 10 mg/ml C) Manganese chloride concentration at 20 mg/ml.

FIG. 19 shows examples of experimental set up for ultrasound testing of nanoshells. Nanoshells were suspended in the sample holder at 400 ug/ml. The imaging transducer and the HIFU transducer were aligned orthogonally.

FIG. 20 shows examples of Color Doppler Ultrasound Testing of nanoshells. A) Mean MI threshold to generate Color Doppler signal, the error bars signify standard deviation. B) Imaging lifetime in minutes of nanoshell formulations with continuous color Doppler imaging at MI=1.9, error bars signify standard deviations. *Note that the Phenyl and C8 particles had no standard deviations because the test was terminated at 180 minutes. C) Color Doppler signal from C8 nanoshells detected at the initiation of the continuous imaging study. D) Color Doppler imaging of the same C8 suspension at 180 minutes. Note that although the signal decreases after 180 minutes, it was still robust. The long imaging times of the C8 and phenyl particles correlate with these having the thinnest shell walls as shown in SEM and TEM imaging.

FIG. 21 shows examples of image brightness over time with CPS imaging as a function of MI of PFP gas filled nanoshells. A) Signal brightness plotted with mechanical index as a function of time (frames) for control nanoshells. B) Signal brightness and mechanical index as a function of time (frames) for phenyl nanoshells. Note that with each MI step, brightness increases and then decays until the next MI step. Also note the greater signal generated with the Phenyl formulation at each MI and the slower decay even at maximum MI of 1.9. C) CPS image of control nanoshells acquired at MI=1.5 D) CPS image of control nanoshells acquired at MI=1.9. Note the greater brightness at 1.9 MI. E) is a plot of peak brightness on CPS images as a function of MI for all formulations. Note that the Phenyl formulation had a relatively low threshold and generated the brightest signal at all MIs. Note: The sample gas volumes occupied between the nanoshells and Definity™ are equal; however; the Definity™ particle count is 64 times less than the nanoshells due to their larger size. F) Pressure threshold of the HIFU pulse that generated signal on the CPS image. Error bars signify standard deviations, N =5.

### DETAILED DESCRIPTION

Disclosed are methods and materials pertaining to silica nanostructure, including silica nanoshells and nanoflakes, versatile, large-scale manufacture methods to fabricate silica nanostructures, and examples of applications of the nanostructures.

The disclosed technology includes silica nanomaterials with engineered structures including nanoshells and nanoflakes. The disclosed methods include a variety of nanoscale fabrication techniques, including, for example, a technique for facile large-batch doping of sol-gel synthesized particles; a technique for scale-up synthesis of silica nanoshells based on stirring, a low-cost, aminated-silane-assisted template technique to synthesize silica nanoshells; and a scalable technique to synthesize tunable nanoflakes and nanoshells assembled from nanoflakes. The disclosed nanostructures are useful in a variety of applications, e.g., such as ultrathin silica nanoshells for ultrasound contrast agents, high frequency induced ultrasound ablation of tumors, and silica nanoflakes as interlayer dielectric in graphene-insulator-graphene devices.

### Nanoflake Synthesis

It is known that silica nanoparticle growth can be modeled using a LaMer model of growth. The LaMer model states that at a critical free energy, nanoparticles of a certain critical radius begin to form, which depends on concentration of precursor and other parameters. For silica, nanoparticles may result from either continued growth of nuclei or by agglomeration of smaller nuclei. By utilizing a sufficiently high shear force from mixing, it is possible to encourage the growth of 2-D nanoflakes rather than the growth of 3-D nanoparticles. The term nanoflake has been typically used to describe nanomaterials which are plate-like with dimensions ranging from 1 nm-500 nm in diameter, but less than 10 nm in thickness. Nanoflakes can be synthesized from a wide variety of materials and for a large spectrum of applications, e.g., such as improving electronic properties of materials or modifying thermal properties. In layer by layer deposition during sol gel reactions, nanoflake precursors may lead to more dense and compact structures.

In one example of the conventional technology, 100-200 nm diameter spherical ceria (CeO₂) nanoparticles synthesized for catalysis were composed of an aggregate of nanoflakes. The nanoflakes were square in structure and approximately 10 nm by 20 nm. For example, ceramic nanoflakes that were primarily silica based can be synthesized by hydrothermally treating soda lime glass. Such generated nanoflakes were 200 nm in diameter but only 4 nm thick. For example, MnO₂—NiO nanoflakes were synthesized into tubular arrays, which acted as a pseudocapacitor electrode. These nanoflake based nanoarrays were synthesized using existing ZnO nanoarrays as a sacrificial template for deposited KMnO₄ and Ni(NO₃)₂. A real capacitance of these structures was improved by four orders of magnitude, as compared to carbon-based psuedocapacitors. For example, pure Ni(OH)₂ heirarchical nanostructures composed of ultrathin nanoflakes were used as materials for pseudo-capacitors. For example, by hydrogen treating WO₃ nanoflakes, nanoflakes were generated to have increased photoactivity, e.g., at least an order of magnitude increase in photocurrent density compared to pristine WO₃. These nanoflakes were synthesized by a seed mediated solvothermal method derived from WCl₆ for the seed formation and Na₂WO₄ for the flake growth. For example, TiO₂ hybrid nanoflake/microparticle structures were shown to improve photocatalytic activity which is derived from the nanoflake nanostructure. For example, upon photoexcitation of these structures, the photocatayltic activity of these particles was shown to aid in the degradation of bisphenal A (BPA) and methyl orange, e.g., demonstrating that these particles could be used environmental applications.

In some implementations of the disclosed technology, a scalable method to synthesize silica nanoflakes using sol-gel techniques is disclosed. The methods disclosed herein entail synthesizing silica and doped silica nanoflakes through sol-gel chemistry yielding nanflakes in the 2 nm-50 nm size regime. The technique used herein is a liquid phase technique that is readily scalable and adjustable for a variety of materials, which are prone to undergo sol-gel reactions. This technique allows for large batch synthesis of sol-gel generated nanoflakes, where size can be controlled by how long the reaction is allowed to proceed before a quenching/stabilizing agent is added. In addition, the solvent concentration and the temperature can be used to alter the nanoparticle before quenching/stabilization. This allows for a wide spectrum of materials in a large range of diameters to be generated using the methods disclosed herein. For example, iron doped silica nanoflakes can be synthesized with a 2D structure in the size regime of 2 nm-20 nm diameter and 1 nm-2 nm thickness. It is within the purview of one of ordinary skill in the art to modify the methods disclosed herein to make larger diameter flakes. The diameter of the flakes is controlled with the introduction of a capping agent which quenches the growth of the particles and prevents Oswalt ripening from occurring by stabilizing the particles to further sol-gel condensation.

In some embodiments, a non-templated, sol-gel reaction is performed under vigorous (high Reynolds number) turbulent mixing. As the nanoflakes begin to form, a chemical capping agent is added, which quenches the sol-gel reaction and prevents particle growth past an experimentally determined size regime.

The nanoflakes disclosed herein have the advantages of, for example, high surface area, low cost of synthesis, and potentially diverse chemical composition. The nanoflakes can be used in a wide variety of applications, such as: modifying electrical properties of materials, modifying optical properties of materials, improving mechanical properties of materials, high surface area catalysis, filtration, drug delivery, biomedical imaging, low K dielectric films in logic semiconductor especially interlayer dielectrics in 2D semiconductor tunnel FETs, and diffusion barrier layers in logic and memory semiconductor chips.

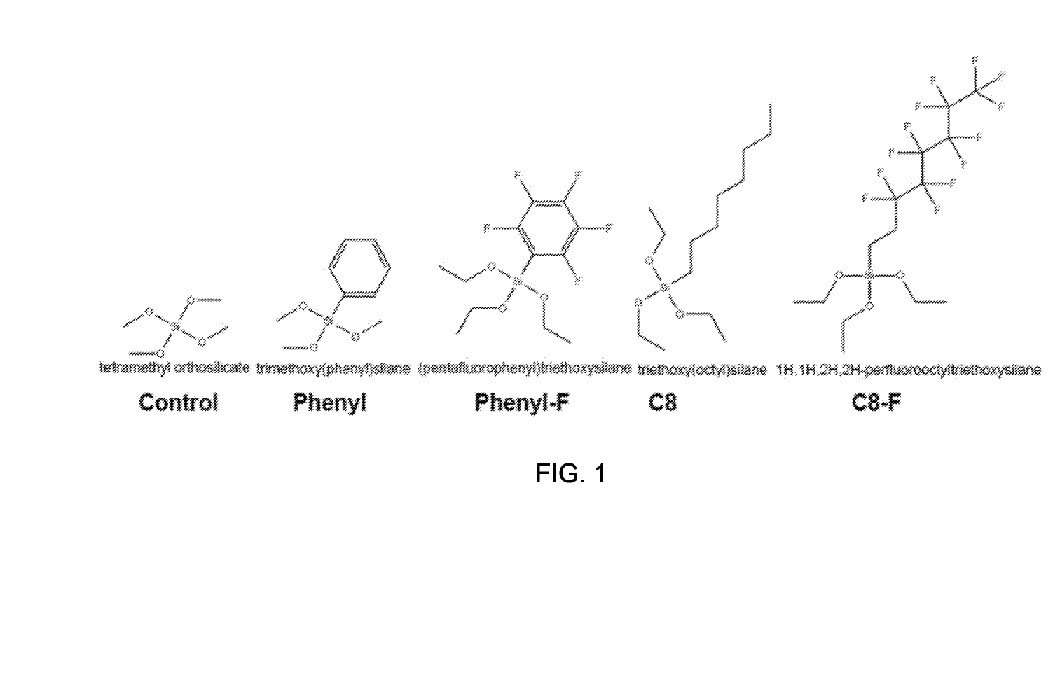

Utilizing organically modified trialkoxysilanes as illustrated in FIG. 1 in the synthesis of nanoshells can result in nanoshells with dramatically improved ultrasound characteristics. An examination of such nanostructures with TEM imaging showed that these example nanoshells were constructed of an assembly of nanoflakes which fused together to form the shell walls. The size of the nanoflakes is dependent on the specific trialkoxysilane present during synthesis. For example, the individual nanoflakes could be synthesized and isolated independently of the nanoshells.

Also disclosed are methods of creating various structures composed entirely of nanoflakes from sufficient templating or pre-existing architecture. For example a large area array of nanoflakes can be created by coating the area of interest in a polycationic material such as polethyleneimine (PEI) or N¹-(3-trimethoxysilylpropyl)diethylenetriamine (DETA). Coating can be performed by either liquid or vapor deposition of the material which becomes cationic in solutions containing water or other acidic groups. After the coating is performed, the area of interest is immersed in a solution in which the nanoflakes are present or being formed. Since the reaction conditions to form the nanoflakes are identical to synthesizing the nanoshells, the nanoflakes in solution adsorb to the cationic material coated surface by a process similar to formation of the nanoshells. After the array or area of interest is coated in nanoflakes, the area is calcined to create a fused structure by forcing surface silanol groups between individual nanoflakes to undergo a condensation reaction.

### Template Synthesis and Scale Up Synthesis of Nanoshells

In some previous examples, silica on nanomaterials were encapsulated and/or coated by surfactant-mediated growth and water/oil emulsions. One example includes techniques with putting a silica coating a top of a polymeric core is to create a layer or coating to adsorb the silica typically with cationic charge. This both creates a charge attraction for the polycondensating silanes as well as provides a framework for the silica to form upon. For example, using polymers such as poly (diallyldimethylammonium chloride), poly (sodium 4-styrenesulfonate), and poly (allylamine hydrochloride) in order to better facilitate the adsorption/deposition of silica can be performed. For example, the surface of metal organic frameworks can be coated with Polyvinylpyrrolidone (PVP) in order to better deposit tetraethyl orthosilicate (TEOS). For example, the surface of polystyrene latex beads can be coated with poly-L-lysine to create a cationic layer for the deposition of tetramethyl orthosilicate (TMOS). For example, silica can be deposited on to cationic co-block polymers poly(2-(dimethylamino)ethyl methacrylate) (PDMA) coronas and hydrophobic poly(2-(diisopropylamino) ethyl methacrylate) (PDPA) to form a silica core shell micelle type structure. Due to the limited solubility of various polymers under alcoholic or organic conditions, for example, polystyrene templates which had been covalently modified with primary amine groups can be to deposit a combination of TMOS and iron ethoxide. For example, a non-polymeric organic (diethylamine) can be used to facilitate the templating of TEOS onto a nickel-hydrazine nanorods but not onto polymer templates.

Large scale synthesis is a term that can refer to the bulk synthesis of a material. Synthetic processes that are usually termed large-scale synthesis are such that can produce materials for commercial purposes, e.g., rather than just experimental investigation that may not require relatively large volumes. Some synthesis processes for various nanoparticle formulations can include the use of stirring or mixing. For example, in one synthesis process, (˜0.1 g) of 2.2 nm silica nanoparticles can be synthesized through use of microwave irradiation. The particles were synthesized by mixing 100 ml of 3-aminopropyl trimethoxsilane with 400 ml of N₂ saturated aqueous solution containing 18.6 g of trisodium citrate dihydrate. The mixture is then irradiated at 160° C./15 min. In another example of a synthesis technique for copper nanoparticles, 30-65 nm copper nanoparticles were created for use in inkjet-printed electronics. To synthesize these nanoparticles, 1.1 kg of PVP, 400 g of sodium hypophosphite, and 1M copper sulfate are vigorously mixed in 90° C. ethylene glycol. After ˜3 minutes of mixing, the nanoparticles are formed and isolated with centrifugation. In another example, silver nanoparticles can be synthesized by gram-scale synthesis. This is done by mixing 1.7 grams of silver nitrate in 0.5 ml of oleylamine and 4.5 ml of oleic acid.

This solution is degassed at 70° C. and then heated to 180° C. The rate of heating dictates the size of the resulting particles, the faster the heating the smaller the particles. After heating, the particles are cooled and precipitated out with a ethanol/toluene solution. Alternatively, for example, an automated batch reactor with constant stirring at 300 RPM can be utilized for the synthesis of SPIONs, e.g., for hyperthermia therapy. In this process, for example, 40.75 mmol of FeCl₂.4H₂O and 74 mmol of FeCl₃.6H₂O were dissolved in 1.1 liters of ultrapure water and heated to 90° C. Then 60 mL of aqueous ammonia and 12 grams of poly acrylic acid sodium salt and the solution were mixed for 1 hour. After mixing, the resulting particles were collected and purified.

In another example, a method for large-scale synthesis of hollow silica nanoparticles based on the traditional Stober method can be performed. For example, 80 mg of poly acrylic acid, 1.5 ml of ammonia hydroxide in 30 ml of ethanol can be mixed vigorously and then 0.75 ml of 27 wt % TEOS can be added. It should be noted that these particles have a large size distribution and very non-uniform surface morphologies. In some examples, a gram-scale method for silica nanorods can include templating and etching nickel-hydrazine nanorods. The nanorods can be synthesized by first mixing 8.5 grams of Brij 58 in 15 ml of cyclohexane and stirred with 1.9 ml of NiCl₂ and 0.45 ml of hydrazine hydrate for 3 hours. In this time, nickel-hydrazine nanorods can be formed within the Brij 58 micelles. Then diethylamine and TEOS can be added and stirred for two hours after which the nickel-hydrazine core is selectively etched HCl to leave silica nanorods. Another large-batch synthesis example includes a technique for the production of silica-magnetite 100-200 nm nanocomposites for hyperthermia treatments. For example, one method, which generated approximately 20 grams of particles/batch, can include dissolving 83 grams of sodium silicate in 2 L of water and then passing through an ion exchange column. After 1.8 liters of solution are collected from the column, the pH is raised to 12 with TMA. 900 ml of 23 g/L magnetite solution is added to this mixture and the pH is slowly reduced to 10 and stirred for 2 hours. Magnetic sedimentation was then used to separate and wash the particles. Also, for example, a large-scale synthetic technique for the production of silica nanowires which were several millimeters in length but only 50 nm in diameter can include growing the nanowires in a tube furnace by first placing five (100) Si wafers (10 mm×50 mm) and then heating the furnace to 1300° C. for five hours. During this time, argon gas (99.99%, H₂<1 ppm, H₂O<or=20 ppm, O₂<or=20 ppm, hydrocarbon<or=6 ppm) was kept flowing through the tube at a rate of 50 sccm and a pressure of 300 Torr.

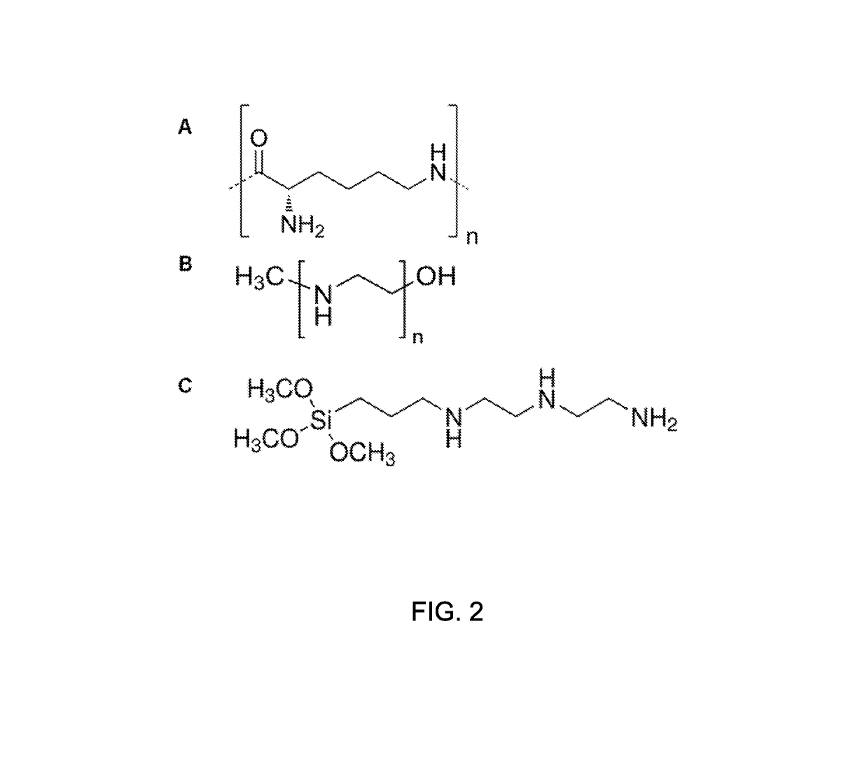

Previous synthetic methods explored required either the use of pre-aminated polystyrene templates or the use of a polycationic polymer to coat the polystyrene template. The aminated layer is necessary to create a framework or starting point for the silica matrix to form during polycondensation. The use of a polycationic polymer is suitable for any aqueous phase synthesis that has been previously performed; however, for the incorporation of various metallic dopants or organically modified silanes, it is necessary for the synthesis to be performed under alcoholic or organic solvent conditions where only small amounts of water are present. However, due to limited solubility of the various polymers in these solvents and the decreased interactions between the polystyrene templates and the polymers, there has been no real success using these polymers under non-aqueous conditions. Furthermore, the production of pre-aminated polymer bead templates are both unreliable and expensive, which provides a barrier to commercial development and production. The disclosed methods include using DETA during the synthesis of these nanoshells as a more reliable and less costly approach. As shown in FIG. 2, the organic R-group structure of the DETA is analogous to that of the polyethlyenimine (PEI), which has previously produced successful nanoshells as an added polymer templating agent. However, the three methoxy groups present on the silicon atom allow for solubility in alcoholic conditions. Furthermore, these methoxy groups undergo polycondensation, allowing for the DETA to self-polymerize on the surface of the polystyrene template leading to both continued polycondensation providing amine nucleation sites on the surface of the nanoshells, as well as a polyamine framework for catalyzing polycondensation of additional silica precursors. Overall, this results in a more reliable and lower cost method of producing nanoshells as it allows the use of common unmodified polymer beads. The disclosed techniques can also be applied to other aminated alkoxysilanes, e.g., including for example, 3-aminopropyl triethoxysilane, 4-amino, 3,3-dimethylbutyl trimethoxysilane, (aminoethylaminomethyl)phenethyl trimethoxysilane, 4-aminobutyl triethoxysilane, N-(6-aminohexyl) aminopropyltrimethoxysilane and many others. Different aminated alkoxysilanes with slightly different structures and varying solubilizes in other solvents may be advantageous for synthesis processes which incorporate materials that are incompatible with alcoholic or aqueous conditions. This technique can be applied to the templating of any negatively charged particle or bead.

Accordingly, in some implementations of the disclosed technology, a scalable method to synthesize silica nanoshells using templating techniques, e.g., aminated-silane templating, is disclosed. In the synthesis of nanoshells, which included the organically modified trialkoxysilanes, ethanol was used as solvent. This differs from some other processes that were performed under aqueous conditions. For example, in other synthetic protocols, unmodified polystyrene beads were coated with a polyamine polymer, such as poly-L-lysine (PLL). However, this approach was not optimal under ethanolic or organic solvent conditions. Polystyrene beads, which were covalently modified to have an amine rich surface, were explored as an alternative. However, the production of these amino-polystyrene beads was expensive, inconsistent, and unreliable. The disclosed templating methods can produce an aminated surface on the polystyrene. One exemplary technique includes the use of the poly-aminated silane N¹-(3-Trimethoxysilylpropyl) diethylenetriamine (DETA), as a co-condensate during the sol-gel synthesis, which can adsorb and copolymerize on the surface of the polystyrene templates allowing for the organically modified silanes to be used in the synthesis of the nanoshells in organic solvents that contain only small amounts of water necessary for the sol-gel reaction.

Therefore, the methods disclosed herein are directed to synthesizing silica nanoshells without the use of a cationic polymer or covalently modified polystyrene templates. This allows for a greater range of solvents to be used, which thereby increases the diversity of possible silanes and other sol-gel precursors, which may have limited solubility.

Exemplary implementations of this example technique have demonstrated greatly lowered cost, scaleability, and increase in consistency, which is extremely important for commercial scale up and FDA approval, e.g., since consistent source materials are required for in vivo biomedical applications in humans.

Large-scale synthesis is a term that can refer to the bulk synthesis of a material. Synthetic processes that are usually termed large-scale synthesis are such that can produce materials for commercial purposes, e.g., rather than just experimental investigation that may not require relatively large volumes.

Conventional methods synthesize silica nanoshells in 2 mL Eppendorf tube reaction vessels using a vortex mixer. The silica nanoshells are actually composed of a hierarchical structure of ˜10 nm nanoflakes as shown in FIG. 1. The size of the nanoflakes is determined by a variety of conditions, such as solution concentration of templates, silica precursors, solvent, etc., but a major factor is also the type of mixing. In order to scale up the conditions similar to those present in the 2 mL Eppendorf tubes under vortex mixing, a non-linear increase is generated in the shear force being produced. If a larger magnetic stirring plate is used, then a wide stir bar is used for mixing. For further scale up of solution volume an impeller stirrer operating at very high speed is used to generate turbulent flow and very high shear force. This modification restricts the growth size of the nanoflakes that are the structural precursors to the nanoshells. If the nanoflakes are sufficiently small, the nanoshells will eventually form given enough assembly time under mixing, if the nanoflakes are too large colloidal amorphous material is recovered.

The growth of nanotechnology and the improved understanding of the needs for both templating and forming of the shells from the nanoflakes lead to the disclosed methods by which to scale the synthesis (e.g., to at least a pilot plant stage) and demonstrate the feasibility for commercial production. For example, synthesizing solid silica nanoparticles (e.g., by the Stober method) was to some degree dependent on the stir rate or shear force applied to the solution. The disclosed techniques can utilize increasingly wider stir bars on a stir plate or a high shear mixer could allow for the synthesis of the nanoparticles by rapid stirring rather than vortexing. This aspect of the disclosed technique allows for increasingly larger batches of nanoshells to be synthesized using inexpensive unmodified polymer beads.

### Silica Doping

In some examples, iron (III) ethoxide can be incorporated into the synthesis of silica particles in order to synthesize biodegradable silica particles. In this iteration of iron incorporation, the iron can be added at the same time as TMOS (silica precursor), which limits the amount of iron that can be incorporated into the particle as the rate of hydrolysis of the two components differ. In some examples, europium can be doped into titania and silica particles to create composite particles, e.g., which could be used for 2-photon imaging due to the presence of small amounts of europium. In these syntheses, the europium was also added at the same time as the silica/titania precursor, but the amount incorporated was limited to ˜1% in template syntheses due to the very differing reactivities of the dopant and silica or titania precursors. In some examples, boron can be doped into silica shells in order to mechanically reinforce the silica shells to be able increase the diameter of the hollow shells without dramatically increasing the thickness of the shell. In the synthesis, trimethyl borate was added after the TMOS precursor but during the time that polycondensation of the TMOS was occurring. In some examples, nickel-silica particles can be synthesized by performing sol-gel with TEOS on top of nickel nanoparticles and then performing hydrothermal treatment on the resultant particles to generate nickel phyllosilicate particles. After calcination, the nickel phyllosilicate particles transformed into what appeared to be silica particles functionalized with nickel particles on the surface. It is note that all of the aforementioned techniques were single step depositions and the doping levels achieved were low.

In some examples, a synthesis technique includes template ion exchange (TIE) which utilized mesoporous MCM-41 type silica incubated in under heating in aqueous solution with manganese acetate. In such examples, the manganese ions can substitute in for the template ion (the lipids used to form the mesopores in MCM-41). The resulting materials retained the manganese after calcination at approximately 2.9 wt. %. For example, the mechanism by which cobalt could be doped into mesoporous silica via the template ion exchange technique [Co(en)₃]³⁺ complexes as the cobalt source can be considered in some techniques. For example, the ratio of ion exchange between cobalt and template surfactant was approximately 1:3 due to not all of the templating surfactant being necessary to balance charges within the pores of silica. In some example, iron can be doped into mesoporous silica derived from either FeCl₃ and (Fe₃Ac₆O)NO₃. After mixing these compounds with mesoporous silica particles under mild heating in ethanol and finally calcination, the resulting particles consisted of 1.6% or 2.7% iron by mass. In some examples, a large variety of metals including Al, Cr, Fe, Co, Mn, Cu, Zn, Ti and Zr into mesoporous MCM-41 can be doped via template ion exchange method in order to develop catalytic nanoparticles. This doping can be performed at room temperature in aqueous media, but the resulting compounds were then calcined at 600 C. The Si/metal ratio can vary between 9-50 for the various metals doped. It is note that all of the aforementioned techniques had a low doping level.

Synthesis and doping of bulk silica and other sol-gel materials as well as the synthesis of various silicate minerals can be performed. For example, silica gels can be synthesized that are doped with europium or terbium derived from TEOS and europium chloride or terbium chloride. In such examples, the Eu or Tb can be first mixed with paraaminobenzoic acid or 1,10-phenanthroline for coordination of the ions before being mixed with TEOS to form the bulk silica gel. The gel can be dried out at 373K and achieved a doping level varying based on initial dopant concentration between 0.5-4% wt. O For example, silica gels can be synthesized that are Er/Yb doped to be used for optical amplification, near IR luminescence, and optical frequency upconversion. In this synthesis, TEOS was prehydrolyzed before the addition of Er(NO₃)₃ or Yb(NO₃)₃ which condensed with the TEOS under acidic conditions to form a gel. Notably, high doping concentrations were not addressed in such techniques. For example, neodymium can be incorporated into silica gels, e.g., for applications in laser technology. For this synthesis, acidified NdCl₃ can be mixed with TEOS in DMF and water and later propylene oxide. The samples can be cast in cylinder tubes and then dried at 160° C. following a series of heat treatment steps ranging from 650-1100° C. The resulting bulk gels had a Nd doping of only approximately 1%. For example, a silica substrate can be doped with Er and Nd with an aerosol based technique. SiCl₄ and XCl₃ can be fed into an atomizer which deposited a soot on a silica substrate which was then sintered at 1350° C. resulting in a 6 micron thick doped silica gel. It is note that all of the aforementioned techniques were single step depositions and the doping levels achieved were low.

Conventional technology results in low doping levels. This disclosure provides a simple method by which metals may be doped into so-gel synthesized nano and microparticles. The resultant particles have the properties of a composite material. For example, particles doped with gadolinium remain in particulate form (i.e., not chelated or agglomerated) but have super paramagnetic characteristics. Some desirable properties include:
(1) Iron doped silica particles may be hollow, solid, biodegradable, ferromagnetic and highly echogenic.
(2) Gadolinium doped silica particles may be hollow, solid, super paramagnetic, radiosensitizing and highly echogenic.
(3) Manganese doped silica particles may be hollow, solid, paramagnetic, and highly echogenic.
(4) Calatylic metal (e.g., Pt, Pd, Ni) doped silica particles may be hollow, solid, and have highly dispersed metal.

In some implementations of the disclosed technology, a scalable method to synthesize nanoparticles using high-yield doping of metals into sol-gel synthesis techniques is disclosed. Doping in different elements into the nanoshells can produce nanoshells with a broader number of applications and material properties. Yet, this presents many challenges in determining unique reaction kinetics for each type of material that was introduced into the synthesis. The disclosed techniques provide a better understanding of the formation of the nanoshells and applies a strategy of integrating new elements into the nanoshells by a new method. In one embodiment of the disclosed method, a technique includes utilizing the large number of boundary layers which exist in the nanoshells due to their composition of stacked nanoflakes. In this approach, nanoshells are immersed into a high concentration of desired elemental salts or elemental alkoxide which can permeate the shells and permanently integrate into the shells after calcination.

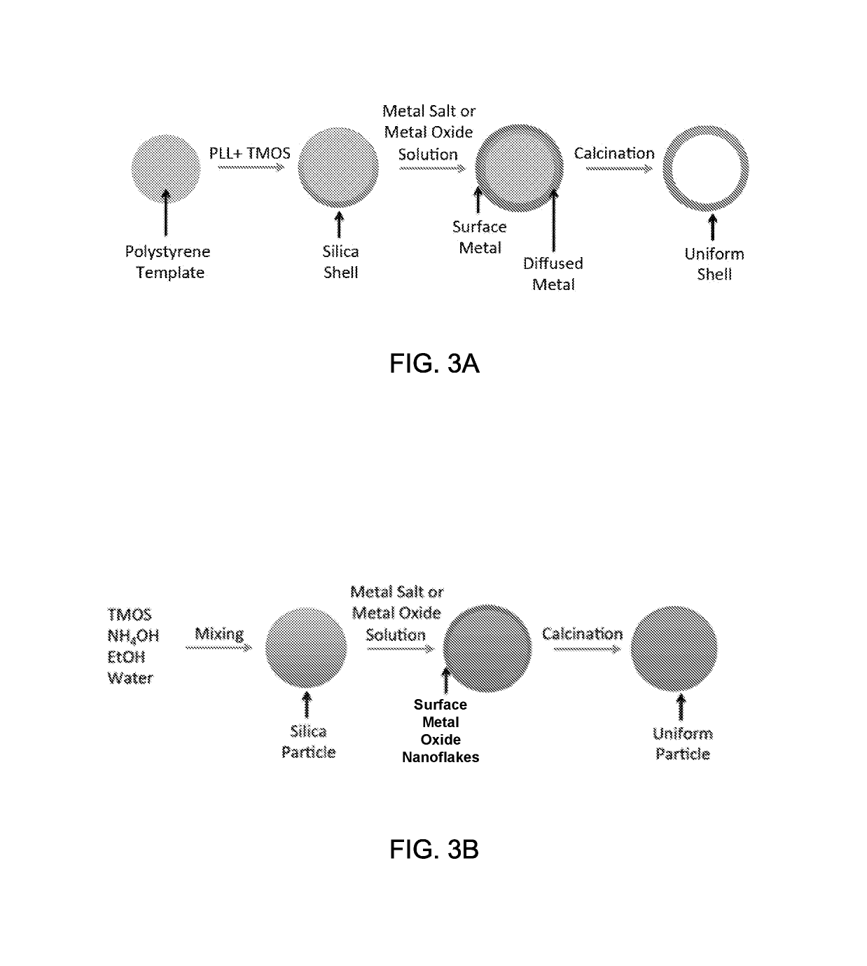

In some embodiments, metals may be doped into sol-gel synthesized nano and microparticles as well as hollow nanoshells as shown in FIGS. 3A and 3B. Some examples of metal particles include 500 nm or 2000 nm particles having different wall thicknesses and some examples of dopants include Fe(OCH₂CH₃)₃, MnCl₂, GdCl₃ and BiCl₃. The doping method disclosed herein can be expended to other metal salts and metal oxides as well as other sol-gel synthesized materials. The doped materials can be any freshly synthesized sol-gel materials not limited to various alkoxy silanes or nano templated materials, but rather any sol-gel synthesized material that is not undergone 100% condensation. The TMOS derived materials exemplified in this disclosure are for illustration purpose only.

This technique allows for facile large batch doping of sol-gel synthesized particles, because it is liquid phase doping that is driven by temperature and concentration. Because of the different rates of sol-gel reactions for the precursor materials, a one-step procedure is not feasible for preparing many doped materials. Unlike other techniques, this process does not require any template exchange or co-doping for high level doping to occur, and the reactions can be performed under mild conditions.

In certain embodiments, doping is done in two stages: 1) synthesis of sol-gel nanoparticles; and 2) incubation of those nanoparticles in a concentrated solution of the metal dopant at various temperatures with continuous mixing. During sol-gel synthesis of silica nanoparticle precursors, room temperature tetramethyl orthosilicate (TMOS) undergoes hydrolysis followed by polycondensation in which the individual monomers form 1 to 4 bonds with other monomers. However, it has been shown that this network is not entirely uniform causing porous particles to form, which results in the particles being permeable to some degree of doping by diffusion. Furthermore, it has been shown that during sol-gel synthesis before the particles are calcined, there are many siloxane monomers which remain unsaturated and partially cross-linked. These can serve to bind or chelate the dopant once it has been introduced into the porous gel. The current particles that have been doped are silica micro and nanoshells. In contrast to the other methods, in one example, before calcination the hydrated particles are soaked in a heated concentrated ethanolic solution of various metal alkoxides, metal oxides, or metal halides (FIG. 3A—Scheme 1). Since diffusion is a function of heating and concentration, the increased heating may aid penetration and reaction of metals into the templated gel. Charge interactions with deprotonanted silanol groups within the particle and the large diffusion gradient may further aid in the retention of the metal ions within the sol-gel and nanoscale sols of the metal oxide precursor added may bind to the surface. Upon calcination, the siloxane network within the particle is known to re-organize, dehydrate and condense. During this process the dopant metal may redistribute throughout the particle, which results in a uniformly doped particle. Nanoscale metal oxide sols that are bound to the surface may also crosslink to integrate into the surface layer. This process also works for small solid particles synthesized by the Stober method as shown in FIG. 3B—Scheme 2.

The doping methods disclosed herein can be implemented in various applications, for example, synthesis of various composite materials, high intensity ultrasound after MRI guided injection of nanoshells, optical materials, use of catalytic metal such as Pt, Pd, and Ni as a support for gas reactions with high flow characteristics, and large-scale doping of sol-gel produced materials to achieve doping level higher than the conventional methods. Additional, the disclosed methods can be used for dual model imaging, ultrasound and MRI for tumor etching and marking. For example, gadolinium and manganese have both been shown to have strong T1 signals, which generates positive contrast under MRI imaging. Gadolinium as used now has several drawbacks, such as risk of kidney toxicity and the need to co-inject a metal ion chelator to minimize risk of Gd interaction with various proteins and the risk of heavy metal toxicity. If the Gd is bound to the particle it may reduce the toxic risk of gadolinium and undergo excretion via the hepatobilliary duct along with the particle. Manganese has only undergone recent investigation for use as a contrast agent and has not been approved by the FDA yet, but integrating it into a particle which has well known toxicology, biodistribution, and excretion pathways will make positive MRI agents with potential much better safety. Note for Fe to work as an MRI contrast it has to be nanocrystalline in a pretty narrow size regime; this is why previous Fe doped particle had no magnetic or MRI properties. Fe derived MRI contrast agents produce a negative contrast which can be useful in regions which would otherwise be too “bright” for visualization.

### Applications of Silica Nanostructures

### 1. Ultrasound Contrast Agents

There are many applications of silica nanostructures, including ultrasound contrast agents. These agents are typically synthesized by encapsulating a perfluorocarbon gas within a lipid or polymeric shell to produce elastic microbubbles in the range 1-6 μm in diameter. Such agents are injected into a patient when preforming the ultrasound imaging procedure. When insonated, these microbubbles oscillate to produce signals at harmonic frequencies and, at resonance, break into smaller bubbles and collapse producing a broadband signal. Since tissues only reflect the primary insonating frequency, contrast specific imaging provides a microbubble only image, which displays the location of the contrast agent on the image. Recently there have been several examples of silica based nanoparticle formulations that can also be used for contrast-enhanced ultrasound. Typically, these particles have been hollow and loaded with perfluorocarbon gas, but there have been examples of solid silica particles that cause increased backscatter compared to tissues. The greatest advantage in using silica nanoparticles as ultrasound contrast agents compared to traditional liposomal/polymeric imaging agents is their improved in vivo stabilities and long imaging lifetimes. For example, gas filled silica particles could be continuously imaged for 45 minutes. Iron-silica nanoshells filled with perfluoropentane could be imaged intermittently over the course of 10 days in a tumor bearing mouse with color Doppler imaging after intratumoral delivery of nanoshells.

It was previously demonstrated that perfluoropentane loaded silica nanoshells fracture on imaging and release the perfluoropentane gas to generate image contrast. Ultrasound imaging power is typically referred to as mechanical index (MI), which is proportional to the peak negative pressure divided by the square root of the imaging frequency. Since silica particles produced greater signal as the MI was increased, it was proposed that there must be subpopulations of particles with varying mechanical strengths that fracture to release the gas at different pressures. As a result, nanoshells are synthesized with a larger percentage of the subpopulations that are mechanically weaker so that the ensemble can be imaged at lower MI for longer times. This was achieved by modifying the nanoshell synthesis with organically modified trialkoxysilanes which produced nanoshells composed of an assembly of ultrathin nanoflakes. The ultrathin nanoshells produced by the modified synthetic procedure had significantly lower color Doppler imaging thresholds, greater imaging longevities, and greater contrast specific imaging performance derived from improved nanostructures compared to control nanoshells made with only TEOS and thus thicker shells.

### 2. Semiconductor Devices with Nanoflakes

Several proposed beyond CMOS devices require deposition of thin insulators between two graphene layers or a pair of 2 dimensional semiconductors such as MoS₂ or WSe₂. Complementary metal-oxide-semiconductor (CMOS) technology, which is based on Si, has been realized for memory and logic devices, due to outstanding performance, reliability, and low product cost. However, as CMOS devices are scaled down to several nanometer dimensions, large leakage current through dielectric are induced, which result in high power consumption. In order to develop alternative low voltage devices with less off-state leakage, graphene has been explored as channel material. Graphene has high carrier mobility and high stability; therefore, most research has been focused on graphene devices based on in-plane transport. However, due to the zero band gap of graphene, integration of graphene into devices with in-plane charge transport results in high off-state leakage current.

Vertical junction with graphene/insulator/graphene(GIG) devices, which rely on charge transfer out of the graphene plane, offer potential low off-state power consumption. Bilayer pseudo-spin field-effect transistors (BiSFET), symmetric graphene tunneling FETs (SymFET), and other 2D tunneling FETs have been proposed. Although the layout of each device is different, all device models are based on the vertical GIG structure. In order to fabricate these vertical GIG devices, an ultrathin (subnanometer or 1˜2 nm) high quality insulating layer should be inserted between two graphene layers and this insulting layer should have modest dielectric constant. Mechanically exfoliated hexagonal boron nitride(h-BN) has been shown negative differential conductance(NDR) at low bias range between two graphene layers. However, due to poor reproducibility of NDR in mechanical assembled G-hBN-G devices, there are still only few reports of their properties. Moreover, mechanical assembly of stacks of exfoliate materials with rotational alignment are unrealistic for large-scale device production. In order to overcome these challenges, ALD (atomic layer deposition) dielectrics have been proposed as the insulting layer. Graphene TFETs based on ALD dielectric have been demonstrated with a low subthreshold swing. However, most research with ALD on graphene has been focused on relatively thick gate oxides (over 10 nm), rather than thin layers, due to large pinhole densities. For low voltage GIG FETs, insulting dielectrics should be less than 2 nanometers thick, which requires nearly uniform nucleation in each unit cell to minimize leakage current.

SiO₂ nanoflakes can be an ideal interlayer dielectric in GIG devices as well as ultrathin interconnect dielectrics in conventional CMOS. SiO₂ has a low dielectric constant as well as a large band gap and large dielectric breakdown. The nanoflakes produced by the methods disclosed herein are about 1.7 nm in thickness, which is close to desired thickness.

The flake diameters are small, 8-10 nm, but larger flakes can be formed. For example, ceramic nanoflakes can be synthesized that are primarily silica based by hydrothermally treating soda lime glass. The generated nanoflakes are 200 nm in diameter but only 4 nm thick. A sheet of nanoflakes can also be assembled on a surface of opposite charge of the nanoflake using hydrodynamic thinning. For GIG devices, instead of assembling the nanoflakes onto a round template for nanoshells they can be assembled on the flat bottom layer (graphene or 2D semiconductor) in the GIG structure. For interconnect dielectrics, the nanoflakes can be assembled on the complex structures now employed in MOSFETS and memory devices. The small size of the individual nanoflakes is advantageous in interconnect dielectrics in conventional where feature sizes are less than 20 nm.

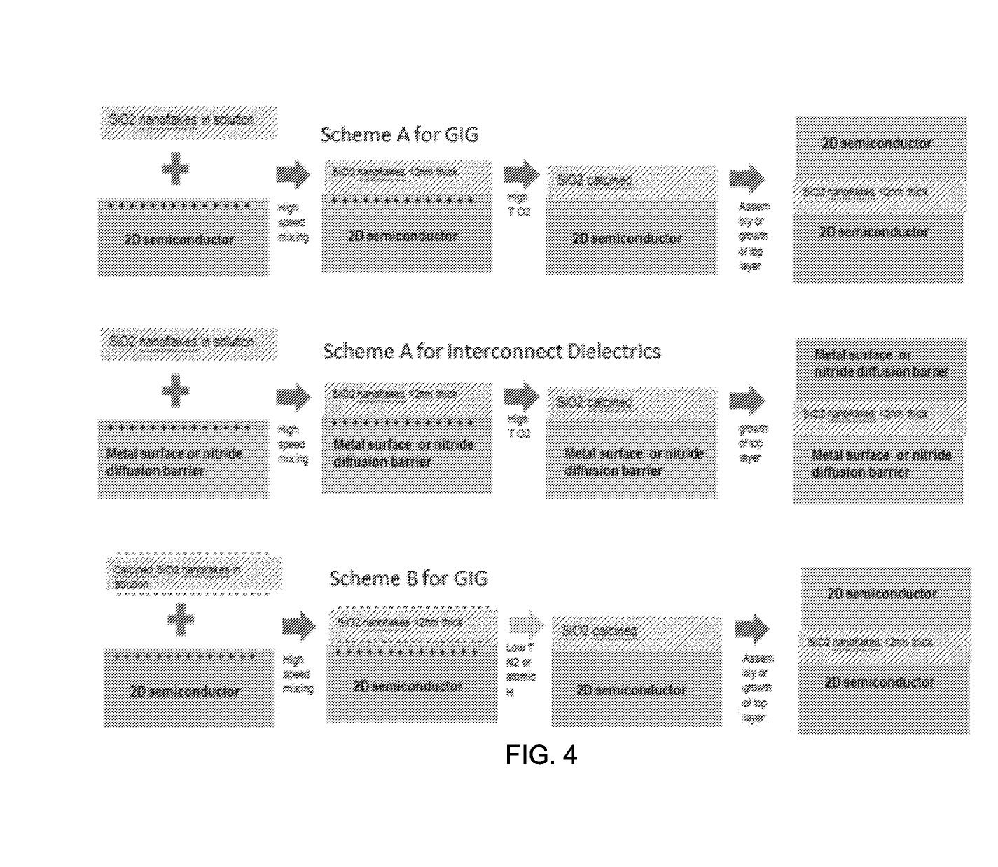

Two variations are disclosed herein, as shown in FIG. 4. Scheme A shows that a 2D semiconductor is coated with a passive charged species, such as a polyamine. The nanoflakes are synthesized using a sol-gel process with tetramethyl orthosilicate (TMOS) and R-substituted trialkoxysilanes at a high enough Reynolds number to limit the thickness of the layer. Afterwards, the nanoflake layer is calcinated at high temperature to produce insulated low defect density silica. Because a high temperature anneal in O₂ is required (550° C. has been tested but 300° C. might be sufficient with longer processing times), this process is suitable for conventional CMOS interconnect dielectrics and GIG made of oxidant stable materials such as graphene but may be challenging for oxidation sensitive materials, such as the 2D semiconductor based on selenium. The templating process can work on nearly any shaped structure so they are ideal for interconnect dielectrics. Scheme B shows that for GIG structures which are temperature or oxidation sensitive, the nanoflakes are synthesized, calcinated, coated with a hydrophilic surface layer, and then resuspended in solution. The bottom layer of the GIG is coated again with a surface species of opposite charge and the nanoflakes is deposited again under high Reynolds number flow to limit the thickness. Afterward the functional groups on both the nanoflakes and GIG can be removed by annealing in an inert atmosphere, or with atomic hydrogen.

The following working examples illustrate various embodiments of this disclosure. By no means the working examples are intended to limited the scope of the invention disclosed herein.

### EXAMPLE 1

### Nanoflakes

Control nanoshells were synthesized. In brief, 50 μl of amino-polystyrene templates were added to 1 ml of anhydrous ethanol. Iron (III) ethoxide was suspended in a second aliquout of anhydrous ethanol at 20 mg/ml. 10 μL of this solution was mixed with 2.7 μl of TMOS, and the entirety was added to the template solution. The mixture was mixed for 5 h, centrifuged and washed twice with ethanol and left to dry in air overnight. The dried particles were calcined for 18 h at 550° C. The yield per Eppendorf tube of final product ranges from 400 μg to 800 μg and typically the products from 24 eppendorf tubes are added to a crucible for calcination. The synthesis of other formulations was similar to that of the control nanoshells. The molar quantity of silicon precursor was kept constant; however, only 30% of it was derived from TMOS and the remaining 70% was derived from one of the following R-group substituted trialkoxysilanes: triethoxy(octyl)silane “C8”, trimethoxyphenylsilane “Phenyl”, 1H,1H,2H,2H-perfluorooctyltriethoxysilane “C8-F”, and (pentafluorophenyl)triethoxysilane “Phenyl-F.” Structures of the R-group substituted trialkoxysilanes additives can be seen in FIG. 1. For the new formulations, R-group substituted trialkoxysilanes were mixed with TMOS and iron ethoxide before addition to the template solution. The nanoshells derived from the “Phenyl” and “C8” were shown to have dramatically improved ultrasound properties. Further investigation by transmission electron microscopy (TEM) revealed the source of this improvement.

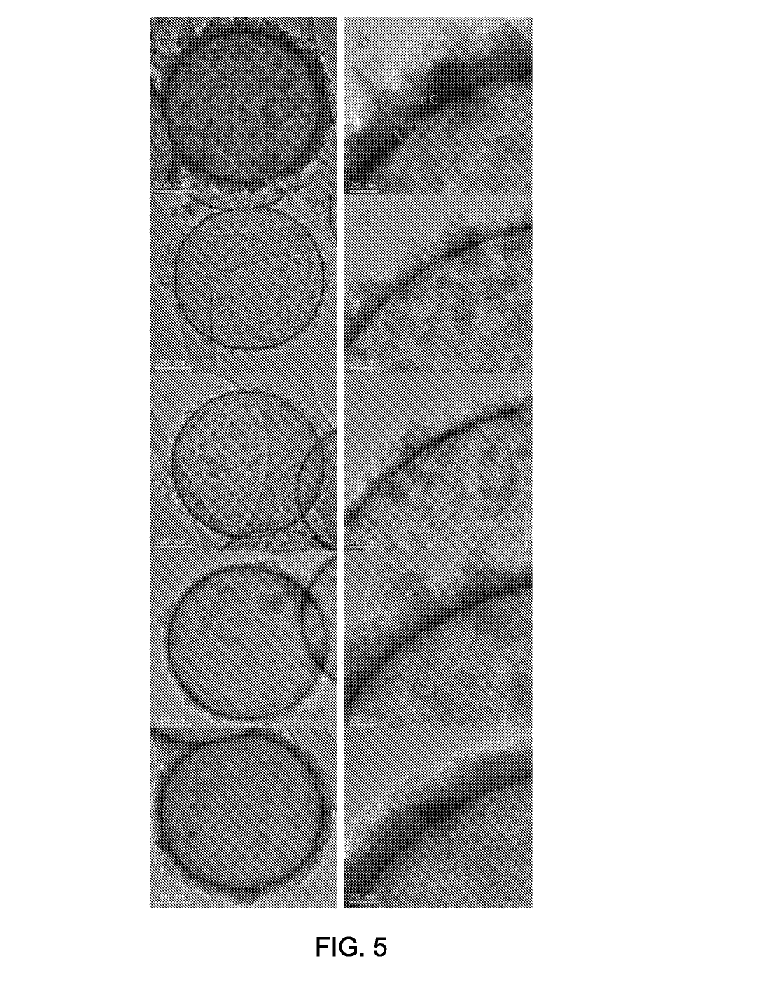

The images in FIG. 5 are consistent with all formulations of nanoshells being composed of fused silica nanoflakes, which are linked together by condensation of surface silanol groups after they assemble on the template surface. These nanoflakes are formed initially during the sol-gel process and are present before calcination. As shown in FIG. 5, the formulations synthesized with R-substituted trialkoxysilanes had markedly thinner shells than the control formulation and, therefore, are denoted as ultrathin nanoshells. There are at least three distinct regions in all of the nanoparticles. The inner dense layer is the black ring seen in images of all the particles (Layer A). The second layer is an intermediate less dense layer between the black ring and the middle loose silica flakes (Layer B). The final layer is an irregular corona of attached exterior silica flakes (Layer C). A diagram of the different layers can be seen in FIG. 5b. The different thickness can play in the ultrasound proprieties. High magnification TEM images were used to further analyze the nanoflakes dimensions. For the nanoflake thickness, it was assumed that the Layer A thicknesses correlate to the thickness of individual nanoflakes. Nanoflake diameters were quantified using the nanoflakes orthogonally aligned to the nanoshells. All the R-substituted trialkoxysilane formulations have decreased nanoflake diameters compared to the control formulations, as shown in Table 1.

TABLE 1
TEM based shell analysis

| | | | | | | | | Min | Max |
|---|---|---|---|---|---|---|---|---|---|
| | | | | | | Nanoflake | Diameter | Flake | Flake |
| | Layer A | Layer B | Layer C | Sum Shell | Sum | Diameter | STD | Diameter | Diameter |
| | (nm) | (nm) | (nm) | Thickness | STD | (nm) | (nm) | (nm) | (nm) |
| Control | 2.8 | 21.4 | 41.3 | 65.5 | 30.2 | 10.8 | 4.7 | 4.4 | 22.8 |
| C8 | 2.7 | 5.6 | 14.3 | 22.6 | 12.2 | 8.9 | 2.6 | 4.5 | 15.6 |
| C8-F | 2.6 | 6.0 | 19.0 | 27.5 | 12.9 | 8.8 | 3.1 | 4.8 | 16.6 |
| Phenyl | 1.6 | 7.3 | 13.1 | 22.1 | 6.5 | 6.5 | 2.0 | 2.4 | 11.9 |
| Phenyl-F | 2.5 | 20.6 | 18.0 | 41.1 | 13.4 | 8.5 | 2.5 | 4.3 | 14.4 |

Twenty measurements were taken for each layer from multiple particles in Image J based on TEM images. Standard deviations for layers can be found in supplemental information. For Nanoflake measurements, fifty measurements were taken for each nanoparticle formulation in Image J from a minimum of four TEM images.

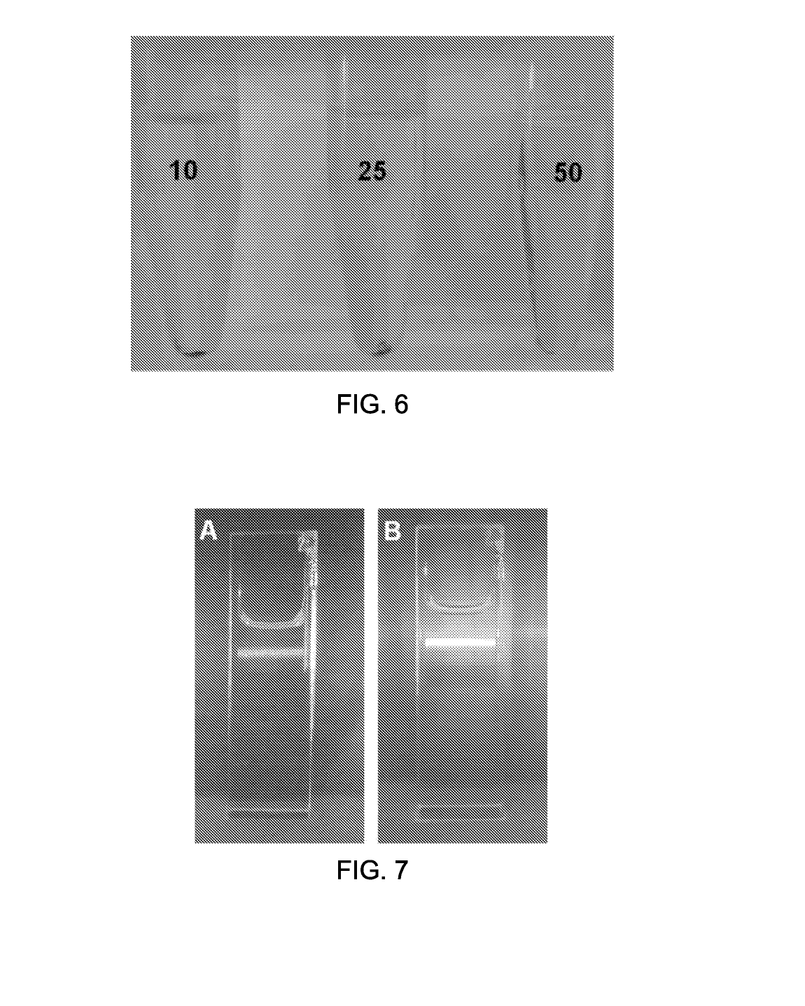

Based on the formation of the nanoflakes in solution and their assembly on the polystyrene templates, the nanoflakes can be independently formed in solution and isolated before they can bind or fuse together. A protocol was used for synthesizing “Phenyl” nanoshells that was adapted to synthesize the nanoflakes. 2.39 μL of trimethoxyphenylsilane (TMPS), 0.83 μL of tetramethyl orthosilicate (TMOS), and 10 μL of iron (III) ethoxide (20 mg/ml) were mixed in 1 ml of ethanol and 50 μL of 0.1% Tween-20 for 2-4 hours. After 2 or 4 hours, 10-50 μL of 1% trimethylchlorosilane (TMCS) in ethanol solution was added to stop the reaction by capping surface silanol groups with the nonreactive trimethylsilyl group. U nlike TMPS which has 3 reactive groups or TMOS which has 4 reactive groups, TMCS only has one reactive group so it acts as a capping agent which halts polycondensation from proceeding. This capping process with TMCS effectively acts as a stabilizer for the nanoflakes as it prevents fusion of individual nanoflakes into larger structures, and allowed for them to be isolated. This effect can be clearly seen in FIG. 6 where increasing amounts of the capping agent added resulted in decreased pellet collection. Only larger aggregated nanoflakes (i.e. nanoparticles) can be centrifuged, while the smaller nanoflakes remain in solution.

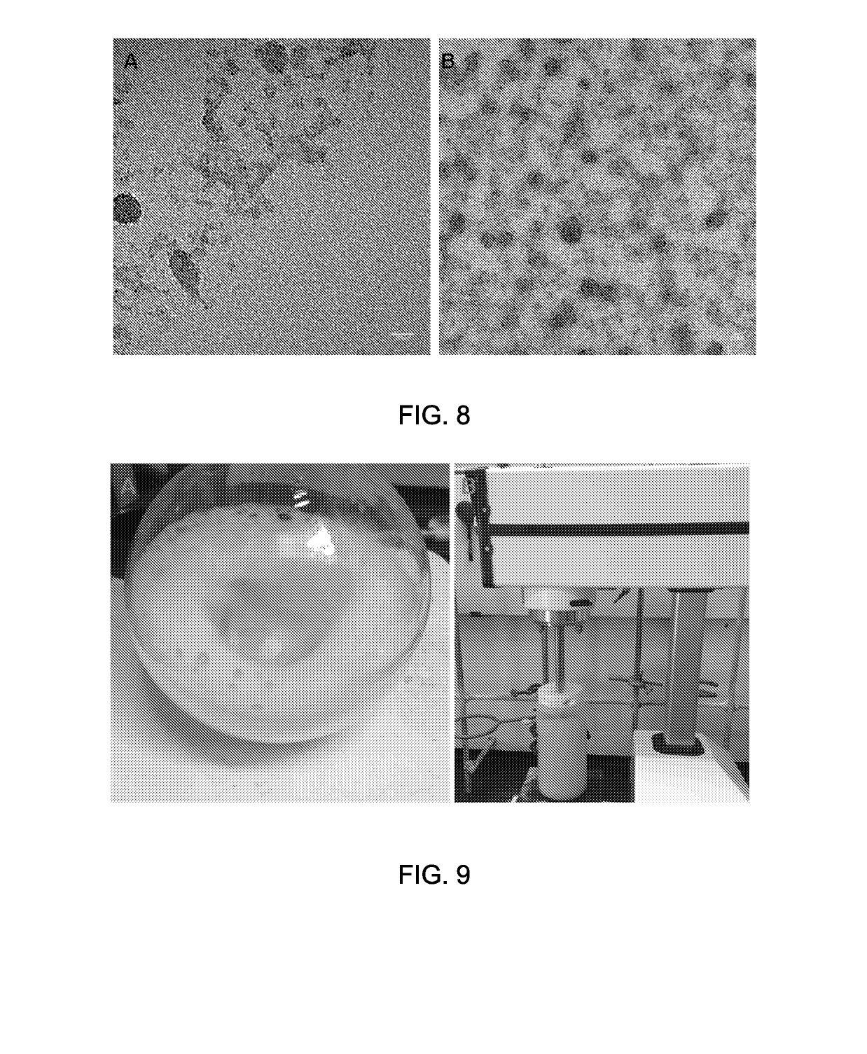

At 50 μL of 1% TMCS addition, no pellet could be observed even at centrifugation speeds of 14,000 G demonstrating that the resulting nanoflakes were sufficiently small that Brownian motion kept them in solution. The first indication of the presence of these nanoflakes was by the Tyndall effect. In FIG. 7, a green laser beam was passed through pure ethanol (FIG. 7A) and through a sample containing the nanoflakes (FIG. 7B). It is clear from this image that the sample containing the nanoflakes had a dramatically increased scattering of the laser which is physically observed as a much brighter beam through the sample. Although the solution was clear to the eye under normal illumination, the small nanoflakes scatter light much stronger than molecules, thereby making the laser beam easily visible as it passes through the solution. To analyze the resulting nanoflakes by transmission electron microscope (TEM), concentrated solutions of the nanoflakes were drop cast onto carbon film. FIG. 8A presents the TEM of the nanoflakes when the sol-gel process was stopped after 2 hours of reaction. The darker features in the 10-20 nm size regime can immediately be seen, but what is critical to notice is that in this image the carbon film cannot be seen. All the visible nanoflakes are actually sitting atop of other nanoflakes due to the high concentration of flakes present. The background flakes are all approximately 2-5 in nm diameter. In FIG. 8B, the sol-gel reaction was allowed to continue for 4 hours which resulted in a larger number of 10-30 nm flakes that are readily observable. This is expected from a longer polycondensation reaction period. Like in FIG. 8A, the larger sol-gel particles also sit upon a bed of smaller 2-5 nm nanoflakes that obscure the underlying carbon film TEM substrate. Nevertheless, the morphological differences between the two reactions ultimately show that in general the diameter of the resulting nanoflakes can be controlled by the length of the sol-gel reaction before quenching. Using the disclosed technology, a variety of nanoflakes can be synthesized from silane precursors under the conditions described. The nanoflakes prepared by this method have approximately equal X and Y Dimensions but very small (˜1-2 nm) plate thickness dimension, Z, resultant from the mixing conditions and quenching of the reaction by capping the OH groups of the growing nanoflakes with SiMe₃Cl or some other terminating group that cannot undergo further sol-gel condensation.

Additionally it is possible to control the deposition and hierarchical structure of nanoflakes with charge interactions. By examining the structure of iron silica nanoparticles, e.g., such as those previously discussed in FIG. 1, and in PCT Publication No. WO2014052911 A1, included as part of the disclosure of this patent document, it is clear that nanoshells are composed of fused nanoflakes. The nanoflakes adsorb to the surface of the polystyrene template due to the electrostatic interactions between the nanoflakes and the polyaminated cationic surface of the templates.

### EXAMPLE 2

### Scale Up

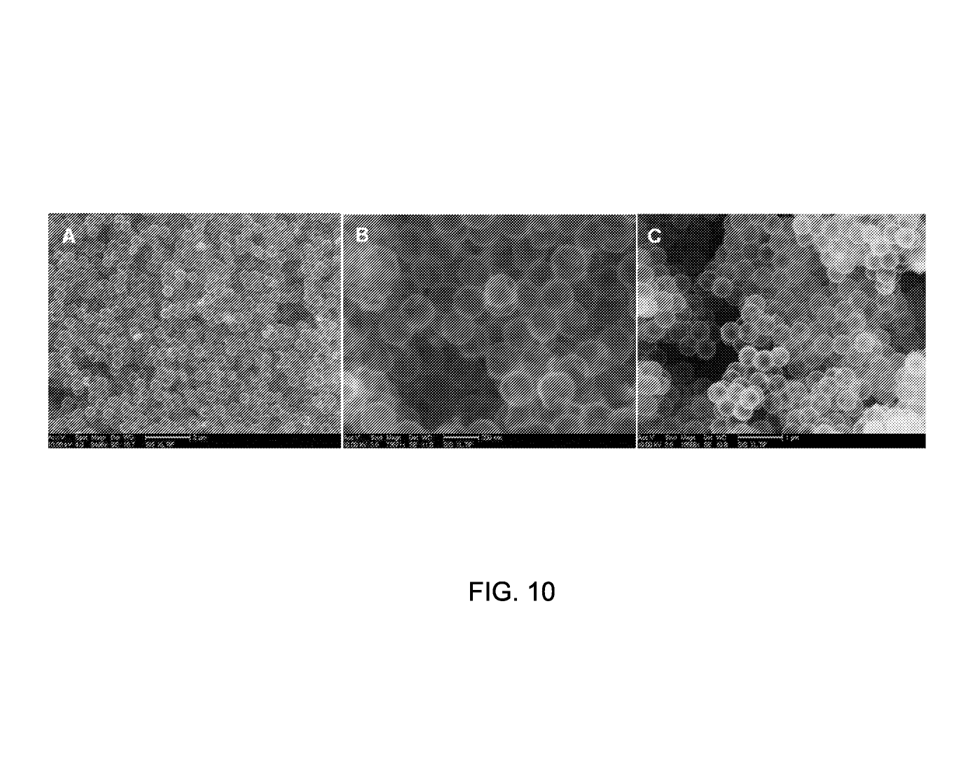

An image of the 100× scale conditions can be viewed in FIG. 9A which shows that mixing is sufficient to cause foaming of the solution from turbulence and the solution is pushed away from the center of the stir bar due to the high speed of mixing. FIG. 10 shows SEM images of the nanoshells resultant from 100× and 300× scale synthesis with diameters of 500 nm and 200 nm, respectively. As can be seen a high uniformity of the nanoshell structure is conserved. In this case, for the 100× scaled nanoshells, the synthetic protocol is as follows. In a 50 mL centrifuge tube, 5 mL of polystyrene templates, 2 mL of 2.5 mg/mL of polyethyleneimine (PEI) and 20 mL of PBS were pulse vortexed for 2 hours. Afterwards, the contents of this tube are added to a 500 mL flat bottom round flask and 80 mL of PBS is added along with a 2 inch stir bar. The mixture is mixed at 1200 RPM. Once the mixing is turbulent, 300 μL of TMOS is added dropwise and the reaction proceeds for 6 hours. After mixing, the solution is centrifuged and the particles are isolated in the pellet and calcined. This process can be performed without the initial pulse vortex step and all steps can be performed in the 500 mL flask. For the 300× process all the volumes are essentially tripled and a 3 inch stir bar is used to achieve the necessary turbulent mixing due to the larger volume of solution being stirred at 1200 rpm.

The scale of synthesis was further increased by moving beyond a stir plate and using a high shear mixer, as shown in FIG. 9B. The high shear mixer poses several advantages, such as higher mixing speeds. This allowed for larger solution volumes, adapting the synthesis to a 1-pot process, and increased consistency due to a fixed impeller head which is more stable at high speed than stir bars. For 500× scale, the synthesis protocol is similar to the 100/300× protocols with several small changes. The 25 mL aliquot of stock polystyrene templates are suspended in 500 ml of 95% ethanol and mixed with the templating solution (DETA/PEI) for 1 hour between 3000-4000 RPM in a sealed Teflon reaction vessel fitted with a custom cap to minimize solvent evaporation; the latter is critical. After 1 hour, the 5 times the equivalent 100× amount of silica precursor (and iron ethoxide) is added dropwise and then allowed to continue mixing for 6 hours. After 6 hours the particles are centrifuged and washed in 1000 ml of ethanol. Following washing the particles are calcined resulting in the material seen in FIG. 10C, which yields between 150-200 mg per batch. The need to incorporate various hydrophobic components such as iron ethoxide or organically modified silanes in larger batch synthesis required the development of a templating method which was compatible with the 95% ethanolic solvent conditions.

### EXAMPLE 3

### Polyamine Silane Templating

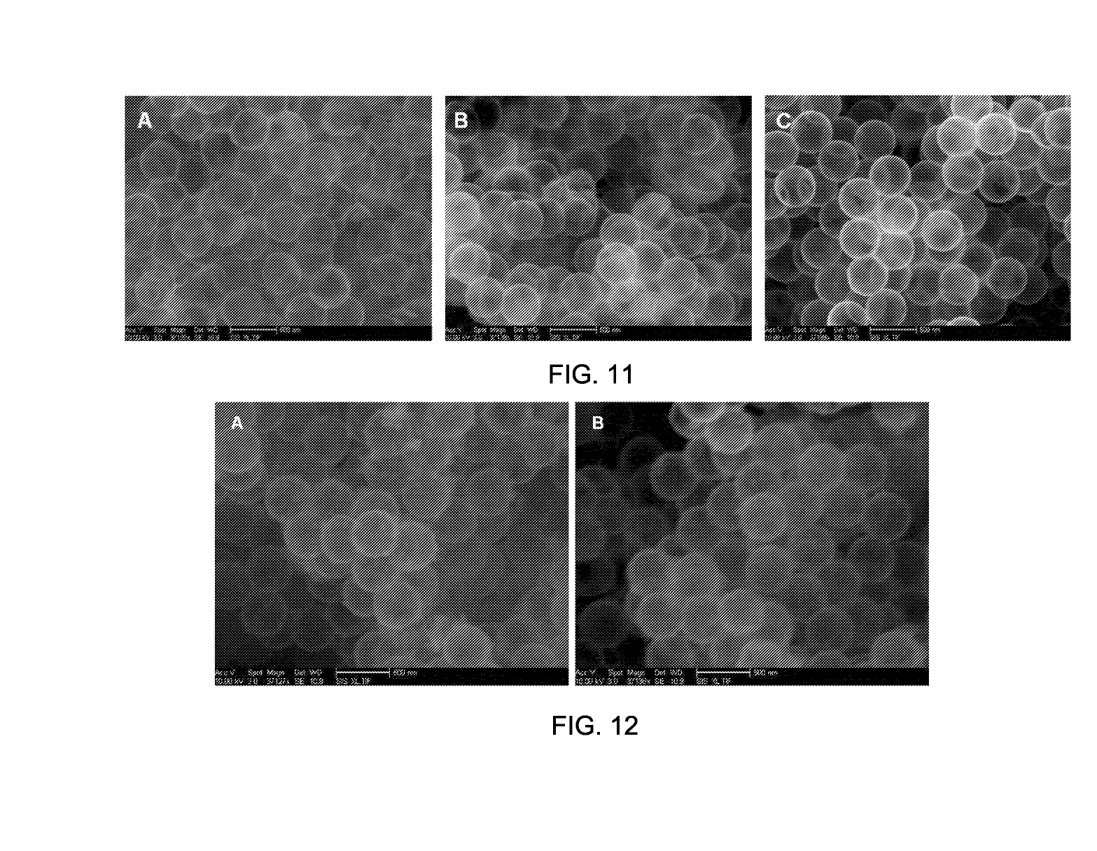

In some exemplary implementations, for example, increasing amounts of 0.2% (v/v) solution were used to determine if plain polystyrene templates could be coated and then reacted with TMOS in ethanol. Briefly 50 μL of unmodified 500 nm polystyrene templates were suspended in 1 mL of ethanol. Then between 30-80 μL of the DETA solution was added and the mixture was pulse vortexed at 3000 RPM for 1 hour. After mixing, 3μL of TMOS is added the mixture is mixed for an additional 5 hours. SEMs of the resulting particles can be seen in FIG. 11, as increasing amounts of DETA was added, the shells appear to be increasingly intact and more robust.

Next iron ethoxide and different organically modified silanes such as trimethoxy(phenyl)silane and triethoxy(octyl)silane were incorporated into the DETA templated synthesis process. As can be seen from FIG. 12, the nanoshells synthesized with organically modified silanes formed robust and uniform shells with iron incorporation comparable to previously values.

To further develop the nanoshell synthetic process, the DETA assisted templating process was combined with the scale up technique and 100× scale was performed to produce larger batches of nanoshells incorporating iron ethoxide and/or the organically modified silanes. For this synthesis, 5 mL of templates are mixed in 14 mL of ethanol with 4 or 8 mL of 0.2% (v/v) DETA solution for 1 hour. Concurrently, the silane precursors are mixed with iron ethoxide in a bath sonicator for 1 hour. The template solution is then added to the flat bottom round flask along with 85 mL of ethanol. This mixture is stirred with a 2 inch stir bar at 1200 RPM. The silane solution is added dropwise and the mixture is stirred for 6 hours. FIG. 12 shows the resulting particles and confirms that they remain structurally similar to the small scale batches shown in FIG. 11. This demonstrates that the DETA templating method can also be scaled up and allows for the incorporation of metallic dopants and organosilanes into the sol-gel synthesis of the nanoshells. These protocols are compatible with the 500× synthesis technique with the high shear mixer. The particles generated in FIG. 10C use a similar protocol to that used to prepare the particles in FIG. 12A, only scaled up an additional 5 times and all performed in a single reaction vessel. 40 ml of 0.2% DETA solution and 25 ml of non-functionalized templates are mixed into 500 ml of 95% ethanol and mixed at 4000 RPM for 1 hour. After 1 hour, the iron ethoxide and silanes are added and allowed to react for 6 hours. After the reaction is complete, the particles are washed and calcined to produce between 150-200 mg of nanoshells.

### EXAMPLE 4

### High Yield Metal Doping

### Stage 1: Synthesis of Hollow 2 Micron and 500 nm Silica Shells

The particles are first synthesized as described in PCT/US2013/062436, the content of which is incorporated by reference in its entirety. 2 micron shells are synthesized by first mixing 2 micron polybead templates, Poly-L-lysine (PLL) and PBS for 30 minutes. TMOS is then added and the mixture is mixed for 7 hours, 2.5 hours into this mixing trimethyl borate in ethanol solution is added. After mixing the particles are pelleted and washed twice with ethanol. 500 nm shells are synthesized by first mixing 500 nm polybead templates, PLL and PBS for 30 minutes. TMOS is then added and the mixture is agitated for 5 hours at room temperature. After mixing the particles are pelleted and washed twice with ethanol. Typically, these particles would then be calcined resulting in hollow particles. 2 micron particles have a shell thickness of ˜40 nm and the 500 nm particles have a shell thickness of ˜15-20 nm. However, for Stage 2 doping, the particles are not calcined until after doping.

### Stage 1: Synthesis of Solid silica particles

Stöber particles were synthesized by mixing 15 ml ethanol, 5 ml water, 0.7 ml NH₄OH and 0.5 ml TMOS for 8 hours at room temperature. After polycondensation of the silica, the particles are washed twice in ethanol and are then ready for Stage 2 doping. The resulting particles are approximately 180 nm in diameter. These are also calcined after Stage 2 doping. Therefore, this is distinct from previous synthetic techniques and the change to a two-step procedure before calcination allows much higher doping levels.

### Stage 2: Gadolinium Doping

To demonstrate the effect that temperature alone can have on the doping of metals into sol-gel synthesized nanoshells, 12 equivalent batches of particles derived from 50 ul of Stock Polysciences 2 micron polybeads and 3 microliters of TMOS per batch were homogenized and washed in ethanol and resuspended in 4 ml of a GdCl₃ (Fe(OCH₂CH₃)₃, MnCl₂, GdCl₃ or BiCl₃) in ethanol solution ranging from 5-20 mg/ml. The solutions were allowed to mix at 1200 rpm at either 50° C. or 75° C. After 48 hours the particles were washed 5× in 50 ml of ethanol and calcined at 550 C for 18 hours. The resulting particles were analyzed by scanning electron microscopy (SEM) and energy dispersive X-ray spectroscopy (EDX).

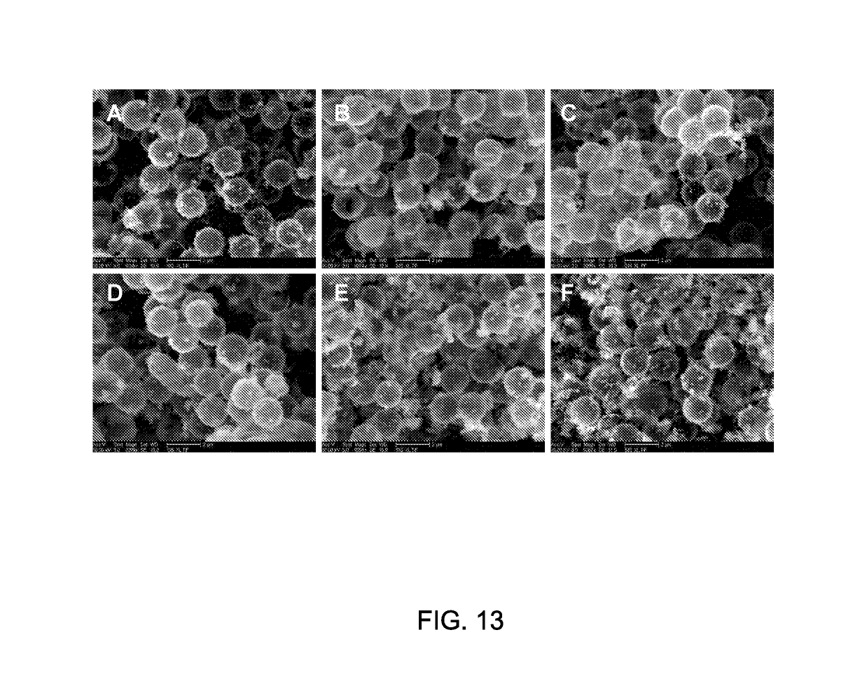

As can be seen from Table 2, as temperature and concentration increased the resulting EDX measurement displayed an increasing amount of Gd content. EDX was performed in three regions and then averaged to generate the averages and standard deviations. As can be seen by the SEM images in FIG. 13, at doping levels beyond 12% there is an increase in the amount of colloidal material surrounding the particles. Part of this colloidal material could be due sol-gel condensation of the gadolinium at higher temperatures and concentrations and might be overcome in the future with longer incubation and lower temperatures and lower concentrations.

TABLE 2
Gadolinium doping into 2 micron shells over 48 hours.

| GdCl₃ Starting | Temperature | Average EDX Gd | Standard |
|---|---|---|---|
| Concentration (mg/ml) | (° C.) | Weight % | Deviation |
| 5 | 50 | 2.4 | 0.16 |
| 10 | 50 | 2.8 | 0.48 |
| 20 | 50 | 9.0 | 2.6 |
| 5 | 75 | 12.2 | 2.0 |
| 10 | 75 | 16.7 | 2.4 |
| 20 | 75 | 16.9 | 2.4 |

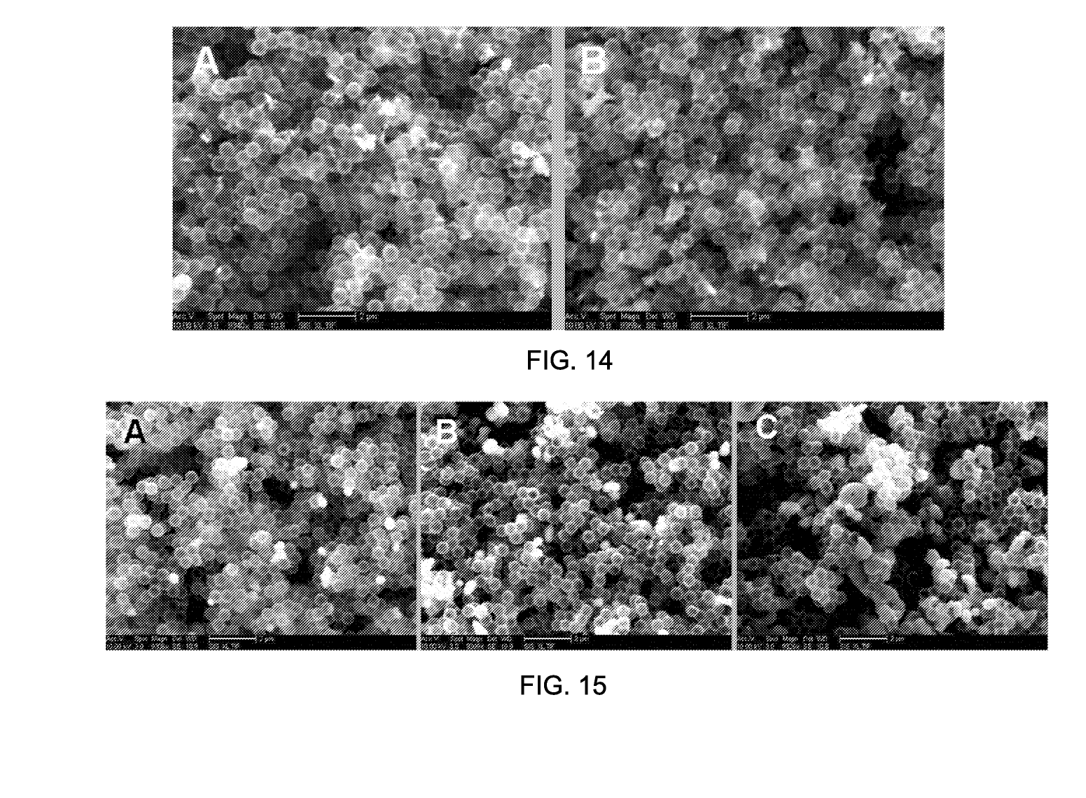

This doping process with gadolinium was repeated for 500 nm particles. FIG. 14 shows 500 nm particles which were synthesized at room temperature then incubated for 48 hours at 75° C. in (A) 10 mg/mL GdCl₃ solution and (B) 20 mg/ml solution. As can be seen from the SEMs there appears to be much less colloidal material on the surface of the particles while the EDX values are similar. This suggests that to some degree the efficiency or uniformity of the doping may be a function of surface area because the net mass of silica between the two micron particles and the 500 nm particles was equivalent.

### Stage 2: Iron Doping

A procedure that is used to generate the gadolinium doped particles can be used to make iron doped particles. In short, the particles that are generated from the initial sol-gel synthesis (Stage 1) are re-suspended in an Fe(OCH₂CH₃)₃/ethanol solution and stirred at 1200 rpm while heating between 50-75° C. As can be seen in FIG. 15, the resulting particles had a range of iron doped into the shells varying from 14.54-43.46% wt. As the concentration of the iron in solution increased, so did the concentration doped into the shells. Additionally, it was demonstrated that the longer incubation times generated particles that had higher degrees of doping.

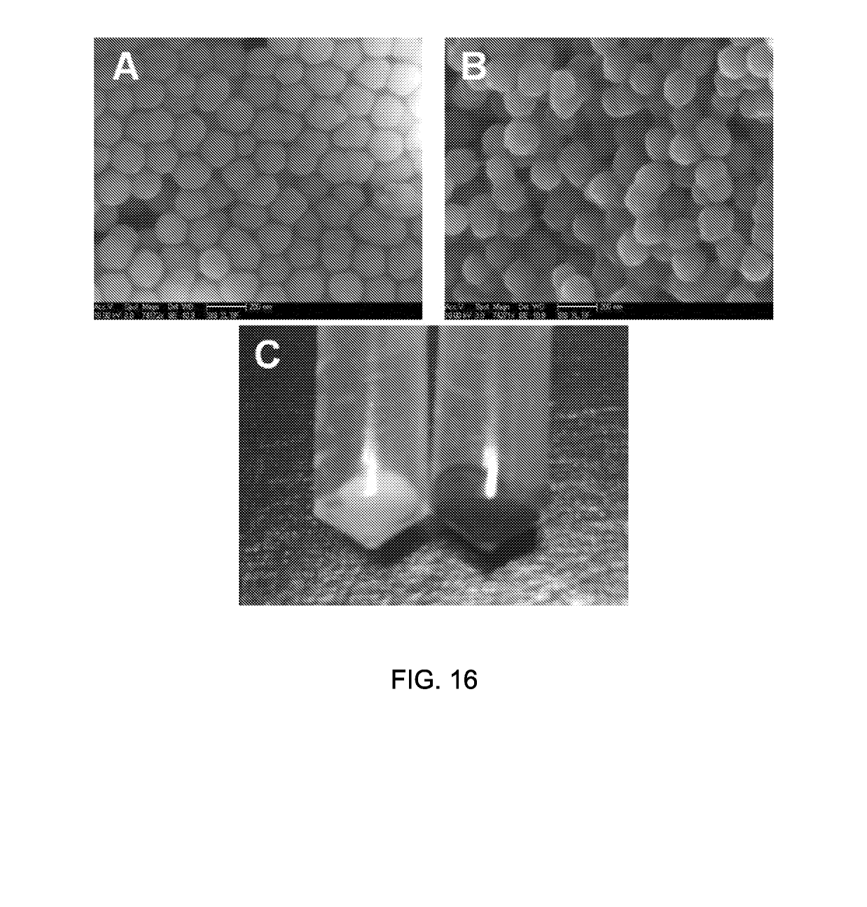

The disclosed two-step doping technique is also applicable to solid silica particles synthesized by the traditional Stober sol-gel synthesis. The resulting particles used in the test were approximately 180 nm in diameter as can be seen from FIG. 16A. These uncalcined particles were isolated and mixed with a 10 mg/mL of iron (III) ethoxide/ethanol solution for 24 hours at 50° C. and can be seen in FIG. 16B. The two samples look very alike under SEM but an examination of the bulk sample in FIG. 16C shows a drastic difference in color due to much high degrees of iron doping attained by the two-step synthetic procedure.

### Stage 2: Bismuth/Manganese Doping

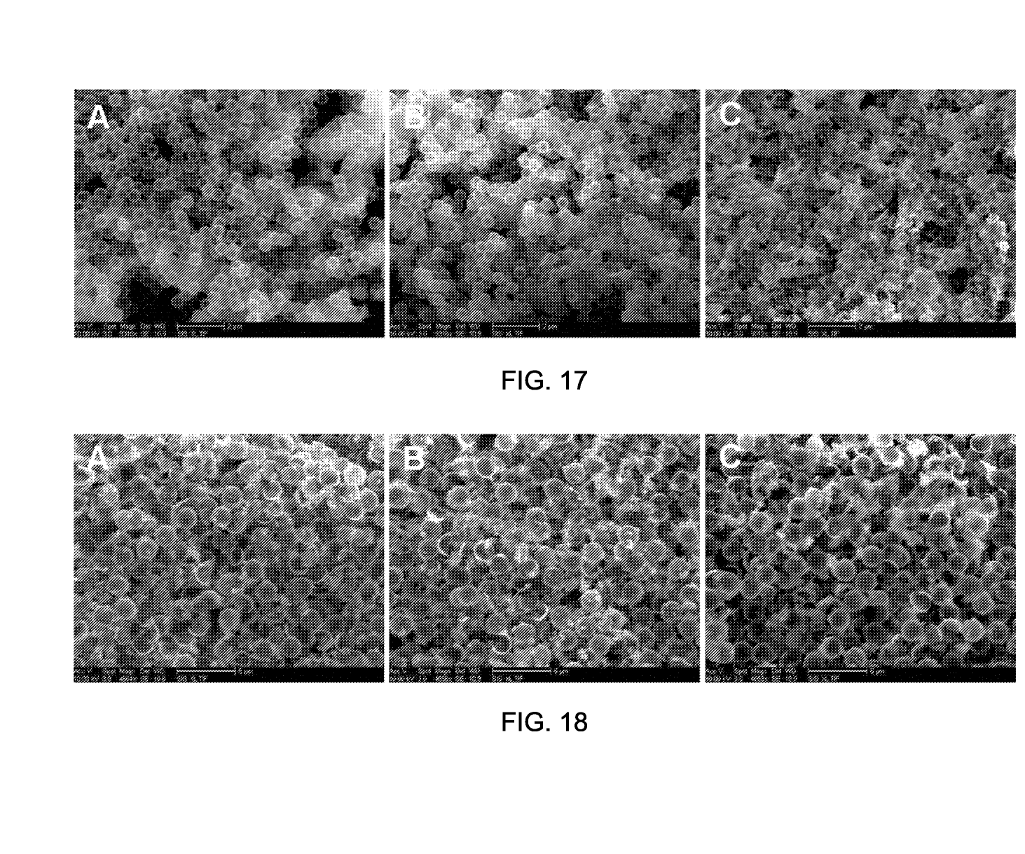

The two-step doping technique has also been applied to doping silica with bismuth and manganese, in example implementations. In such examples, bismuth chloride (BiCl₃) or manganese chloride (MnCl₂) were suspended in ethanol at concentrations ranging from 5 mg/mL-20 mg/mL. Non-calcined shells synthesized at room temperature were incubated in these solutions with mixing and heating (50 or 75° C.) for 48 hours. The particles synthesized with bismuth chloride incubation are shown in FIG. 17. The morphology of the particles remained consistent between incubation with 5 and 10 mg/ml solution (FIG. 17A-17B), but at 20 mg/ml (FIG. 17C) the high amount of doping began to destabilize the particles resulting in numerous fractured particles. A similar effect was observed in FIG. 18 in the doping of manganese into 2 micron silica shells. As the manganese concentration increased, more colloidal manganese was observed to form as a byproduct, as well as an increase in the number of fractured particles. These effects might be overcome in the future with optimized doping conditions or by using particles which are solid or otherwise more mechanically stable; however, limitations may arise from the mismatch of size for Bi(III) and the large charge mismatch for Mn(II). It would be expected that 3+ ions of similar size to Si(IV) would substitute better into the silica gel lattice.

### EXAMPLE 5

### Mechanically Tunable Hollow Silica Ultrathin Nanoshells for Ultrasound Contrast Agents

Tetramethyl orthosilicate, triethoxy(octyl)silane, trimethoxy(phenyl)silane, 1H,1H,2H,2H-perfluorooctyltriethoxysilane, (pentafluorophenyl)triethoxysilane were all purchased from Sigma Aldrich Corp (St. Louis, Mo.). 500 nm amino-polystyrene templates were purchased from Polysciences Inc (Warrington, Pa.). Iron (III) ethoxide was acquired from Gelest Inc (Moorisville, Pa.). Perfluoropentane was acquired from Strem Chemicals (Newburyport, Mass.).

Ultrasound images were acquired with a Seimens Sequoia 512 (Mountainview, Calif.) with an Acuson 15L8 imaging transducer. Sonic Concepts Inc. (Bothell, Wash.) H-102 single element transducer was used to generate high intensity ultrasound pulses that were powered by an AG 1006 Amplifier/Generator (Rochester, N.Y.).

Software used for analysis of data included Sante Dicom Viewer (Athens, Greece), Matlab (Natick, Mass.) and Microsoft Excel (Redmond, Wash.).

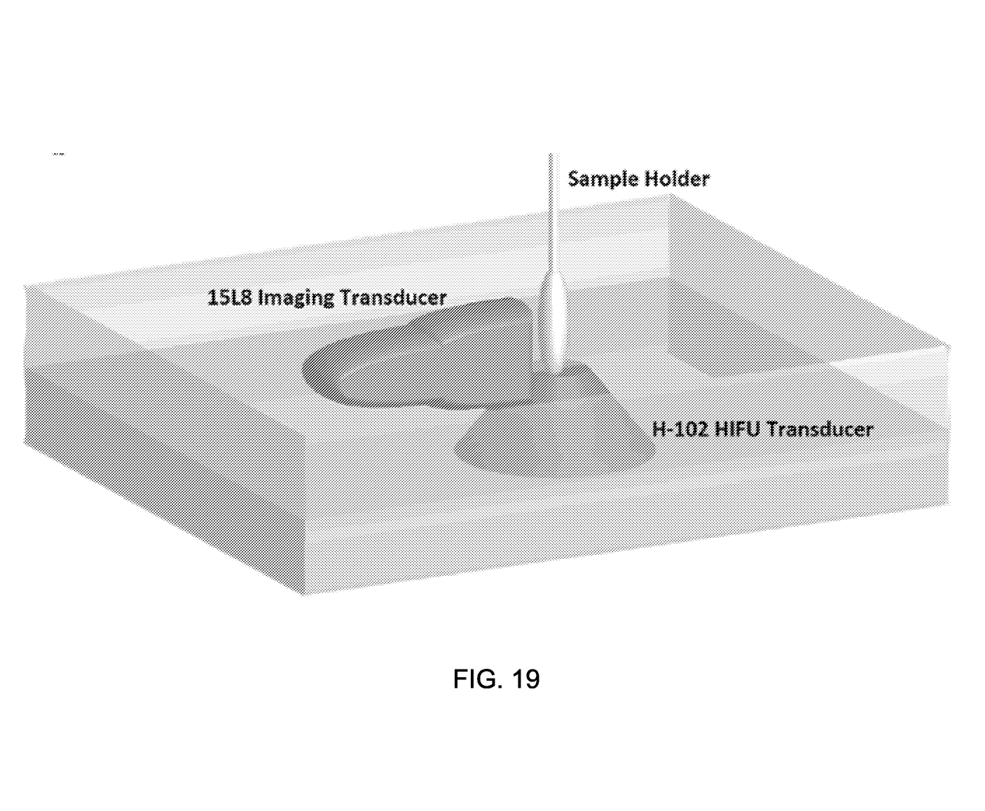

Nanoshells were prepared and SEM analysis were performed as described in Example 1. For all ultrasound experiments, gas filled nanoshells were suspended in a pipette bulb at a concentration of 400 ug/ml. The pipette bulb was clamped perpendicular to the ultrasound imaging transducer, typically on top of the HIFU focusing cone in a water bath, as shown in FIG. 19. Two different types of experiments were performed; the first used the imaging transducer alone. To quantify the sensitivity of the particles to ultrasound, samples were exposed to continuous color Doppler Imaging at 1.9 MI for 180 minutes, or until the color signal could no longer be observed, whichever was shorter. To assess image brightness generation, samples were imaged with contrast pulse sequencing (CPS) at 7 MHz, which was shown to generate the strongest signal from nanoshells. Images were acquired continuously as the MI was increased from 0.06 to the maximum clinically allowable MI of 1.9 to define the minimum threshold for signal generation and to quantify the image brightness produced. Each measurement on Definity™ microbubbles at each MI was done using a pristine sample which had no prior exposure to ultrasound. Peak brightnesses in the CPS images acquired at each MI were recorded. The second experiment utilized the high intensity focused ultrasound (HIFU) transducer for signal generation. This delivered 20 μs bursts at 1.1 MHz, whose pressure amplitude was slowly increased until a contrast signal was detected on CPS images To monitor the particles during the HIFU pulse an imaging transducer was aligned orthogonally and confocally as to not interfere with the HIFU transducer. The imaging transducer was operated in CPS mode at 0.1 MI, which was well below the pressures produced by the HIFU transducer. This technique is denoted as single pulse stimulated imaging.

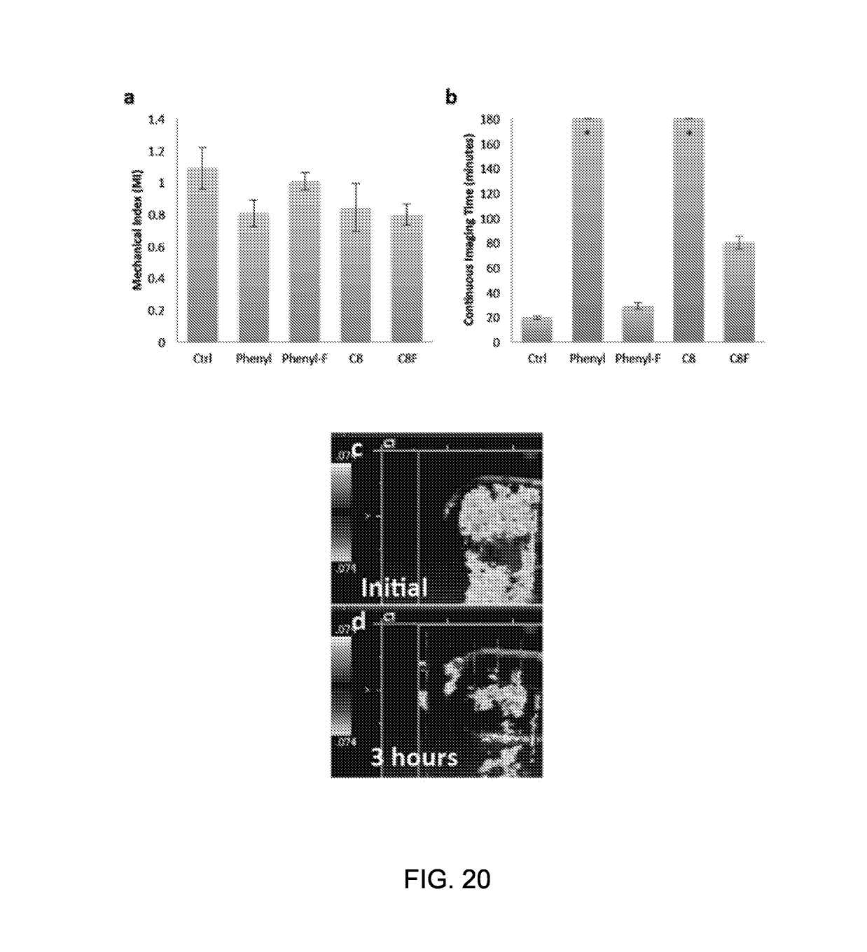

Iron-silica nanoshells were developed as an intraoperative color Doppler tumor marker, and nanoshells were characterized at 7 MHz, the previously determined optimal frequency, for color Doppler imaging threshold and continuous imaging longevity in vitro. As shown in FIG. 20A, the Phenyl, C8 and C8-F formulations had a lower MI threshold compared to the control particles. This is consistent with the particles being more sensitive to color Doppler imaging and consistent with the thinner shells observed for these formulations in SEM. The mechanism for color Doppler imaging of the rigid nanoshells has been shown to arise from nanoparticle fracture and release of entrapped perfluorocarbon gas. To measure the continuous imaging lifetime (FIG. 20B), particles were imaged continuously at MI 1.9 until no signal could be generated from the sample. For the phenyl and the C8 particles, signal continued to be readily observed at 180 min (FIGS. 20C-D). Since the same mass of particle was tested for all formulations, the differences in intensities and imaging lifetimes suggests that they arise from the differences in subpopulations of particles being imaged. It is hypothesized that a greater subpopulation of phenyl and the C8 particles are ultrasound active than control particles.

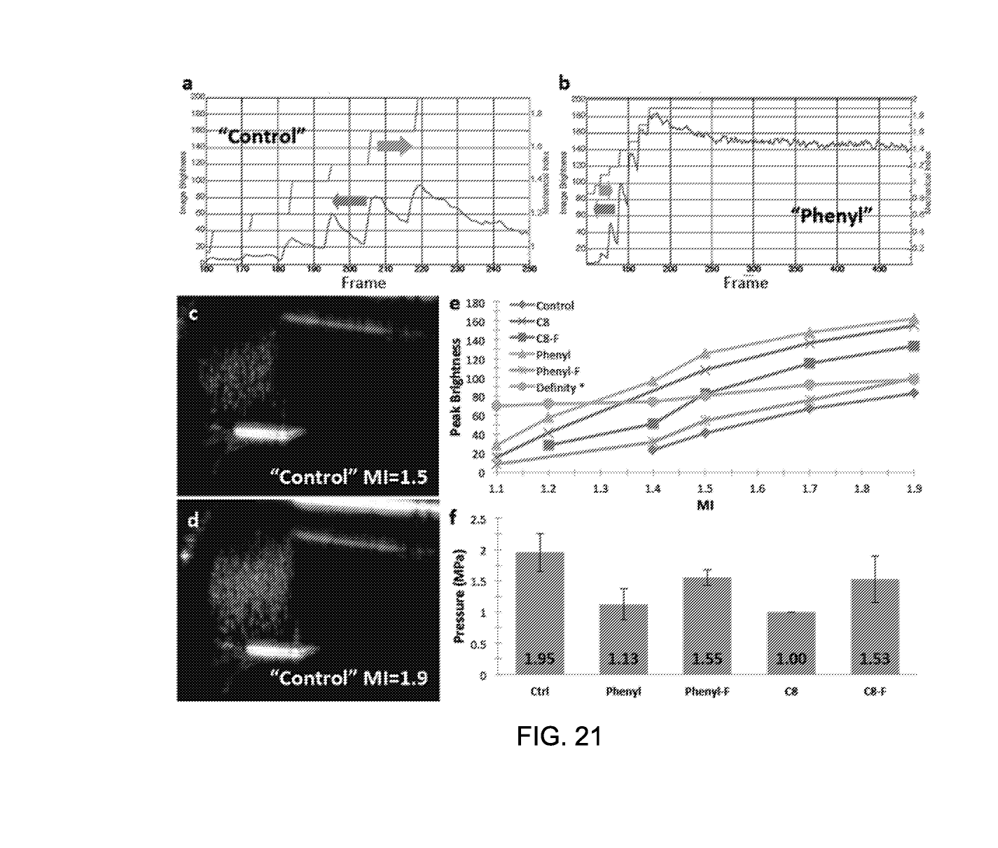

To quantitatively test the hypothesis that a different subpopulation of particles was being imaged at each MI, image brightness on contrast specific imaging was plotted over time (or frame number) for all the formulations; note this is a log scale. FIGS. 21A-B show signal brightness (blue curve) at each MI (green stepwise line) for the control and phenyl nanoshells. Note that signal increases as the MI is increased followed by signal decay as particles of a given sensitivity are consumed. FIGS. 21C-D are representative CPS ultrasound images of the control nanoshells at two different MI settings. Increasing the MI increases image brightness, which is seen as gold speckles on CPS imaging. As shown in FIG. 21A-B, for an equivalent mass of particles, the phenyl particles are approximately twice as bright (on a log scale) at peak brightness compared to the control particles. Furthermore the signal in CPS mode does not experience as much signal decay for the phenyl nanoshells and persists at a brightness of 160 for several minutes compared to an immediate decay to below 40 for the control particles. The difference in imaging persistence between CPS imaging and color Doppler imaging is consistent with the CPS waveform being less destructive than the color Doppler waveform.

FIG. 21E shows peak brightness as a function of MI for each formulation; phenyl, C8, and phenyl-F formulations produced signal at lower thresholds compared to other formulations, and the phenyl and C8 formulations produced the brightest signals. Note that ultrasound signals are log compressed to display the entire dynamic range on the video monitor. Therefore the observed differences in brightness, as shown in FIG. 21, are quite large. Previous reports suggested that the peak-decay behavior of the control nanoshells at varying MI (as shown in FIG. 21A) are consistent with the control particles having different subpopulations with varying mechanical strengths. The present data is consistent with different R-substituted trialkoxysilanes decreasing the average mechanical strength of the nanoshells, thereby allowing a brighter signal as well as an increased imaging longevity. For comparison, commercially available Definity™ microbubbles were diluted to an approximately equivalent gas volume as occupied by the nanoshells and compared directly to the nanoshells. This was done by particle volume because on average individual Definity™ microbubbles have a diameter between 1-3 microns, which results in a 64 times greater volume per particle than the nanoshells. It should be noted that in this MI regime, Definity™ microbubbles are being destroyed similarly to the nanoshells. However, unlike the nanoshell samples where the MI was slowly raised from 0.06 to 1.9 for each sample, each measurement on Definity™ microbubbles at each MI was done using a pristine sample due to the short imaging lifetime of Definity™. An MI of 1.9 is the maximum power used on clinical ultrasound imaging systems. It is evident from this data that the C8 and the phenyl nanoshells generate more contrast at MIs greater than 1.3 than Definity™. This is the first report of any rigid particle demonstrating a signal comparable to a commercially available contrast agent in a contrast specific imaging modality. This illustrates the unique properties than can be generated from nanostructures made of the ultrathin nanoflakes.

To determine the factors that influence the ultrasound signal threshold and intensity, 20 μs pulses at 1.1 MHz were delivered to the sample by the HIFU transducer to fracture and release the PFP gas as the sample was simultaneously being imaged in CPS mode at 0.1 MI to detect the freed PFP bubbles using the apparatus shown in FIG. 19. Note that the 0.1 MI power is well below the signal generation threshold for all the formulations (FIG. 21E). As shown in FIG. 21F, all the formulations synthesized with the R-substituted trialkoxysilanes had a lower pressure threshold compared to the control nanoshells. The C8 nanoshells had the lowest pressure threshold, but this was not statistically different from the threshold of the phenyl nanoshells, both of which had a threshold approximately 50% that of the control. This demonstrates that use of ultrathin nanoflakes can be used for modify the mechanical properties of three dimensional structures.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A method of fabricating a hollow silica nanoshell, comprising:
mixing a particle template with a polyamine polymer and a silica precursor in a solution to coat the particle with a silica shell;
adding one or more metal salts or metal oxides in the solution such that the metal is diffused into the silica shell of the particle; and
calcinating the particle of form a hollow silica nanoshell.

2. The method of claim 1, wherein the polyamine polymer includes poly-L-lysine.

3. The method of claim 1, wherein the polyamine polymer includes poly-aminated silane N¹-(3-Trimethoxysilylpropyl) diethylenetriamine (DETA).

4. The method of claim 1, wherein the particle template includes a polystyrene template.

5. The method of claim 1, wherein the silica precursor includes tetramethyl orthosilicate (TMOS).

6. The method of claim 1, wherein the silica precursor comprises tetramethyl orthosilicate (TMOS) and one or more R-substituted trialkoxysilanes.

7. The method of claim 6, wherein TMOS and one or more R-substituted trialkoxysilanes are mixed at a ratio of 30:70.

8. The method of claim 6, wherein one or more R-group substituted trialkoxysilanes include triethoxy(octyl)silane “C8”, trimethoxyphenylsilane “Phenyl”, 1H,1H,2H,2H-perfluorooctyltriethoxysilane “C8-F”, or (pentafluorophenyl)triethoxysilane “Phenyl-F.”

9. The method of claim 1, wherein the metal oxide includes iron ethoxide.

10. The method of claim 1, wherein the size of the hollow silica nanoshell is between 500 nm and 2000 nm.

11. A method of fabricating a solid sol-gel nanoparticle, comprising:
mixing a silica precursor, in the absence of any template, in an ammonia solution containing ethanol to form a silica particle;
adding one or more metal salts or metal oxides in the solution such that the metal is diffused into the silica particle; and
calcinating the particle of form a solid sol-gel nanoparticle.

12. The method of claim 11, further comprising adding a quenching agent when the silica particle grows to a desired size.

13. The method of claim 12, wherein the quenching agent is a chemical capping agent.

14. The method of claim 1, wherein the mixing is performed at a high shear condition.

15. A method of fabricating a doped nanostructure, comprising:
synthesizing a nanostructure using a sol-gel process; and
doping the synthesized nanostructures by incubating in a concentrated solution comprising one or more metal dopants at a plurality of temperatures with continuous mixing.

16. The method of claim 15, wherein the nanostructure is a hollow nanoshell.

17. The method of claim 15, wherein the nanostructure is a solid nanoparticle.

18. The method of claim 15, wherein the doping is gadolinium doping, iron doping or bismuth doping, or manganese doping.

19. The method of claim 15, wherein the doping is performed in an ethanol solution.
